SEQUENCE LISTING (1 sequence)

SEQ ID NO: 1 - protein, 679 aa, Homo sapiens
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

(19) United States
(12) Patent Application Publication
(10) Pub. No.: US 2025/0290936 A1
(43) Pub. Date: Sep. 18, 2025

(54) METHODS FOR ANALYZING TRANSFERRIN GLYCOFORMS

(71) Applicant: The Board of Trustees of the Leland Stanford Junior University, Stanford, CA (US)

(72) Inventors: Ruben Yiqi Luo, Redwood City, CA (US); Morgan Mann, Redwood City, CA (US)

(21) Appl. No.: 19/079,423

(22) Filed: Mar. 13, 2025

Related U.S. Application Data
(60) Provisional application No. 63/566,813, filed on Mar. 18, 2024.

(51) Int. Cl. G01N 33/68; G01N 1/40; G01N 30/72; G01N 33/543

(52) U.S. Cl. CPC G01N 33/6896; G01N 1/4044; G01N 30/7266; G01N 33/543; G01N 33/6848; G01N 2440/38

(57) ABSTRACT

Provided herein are methods for analyzing a transferrin glycoform, the methods comprising: capturing a transferrin glycoform on a probe that comprises a transferrin glycoform-binding agent; inserting the probe into the interior capillary of an electrospray emitter; releasing the transferrin glycoform from the probe while it is in the emitter using an elution liquid; nebulizing the transferrin glycoform by electrospray; and analyzing the nebulized transferrin glycoform by mass spectrometry. Also provided herein are methods for diagnosing a CSF leak in a subject. The present disclosure additionally provides kits and systems for performing the subject methods.

### CROSS-REFERENCE

This application claims the benefit of U.S. Provisional Patent Application No. 63/566,813, filed Mar. 18, 2024, which application is incorporated herein by reference in its entirety.

### INCORPORATION BY REFERENCE OF SEQUENCE LISTING PROVIDED AS A SEQUENCE LISTING XML FILE

A Sequence Listing is provided herewith as a Sequence Listing XML, “STAN-2192_SEQLIST” created on Mar. 10, 2025, and having a size of 2,588 bytes. The contents of the Sequence Listing XML are incorporated by reference herein in their entirety.

### INTRODUCTION

Cerebrospinal fluid (CSF) leak can occur as a result of laceration, blunt trauma, or surgery and may lead to potentially life-threatening meningitis if left untreated. β₂-transferrin (β₂-Tf) is a proteoform of human transferrin (Tf) that is mainly present in cerebrospinal fluid (CSF) and barely detectable in other body fluids. CSF leak is diagnosed in clinical laboratories by detecting the diagnostic marker β₂-transferrin (β₂-Tf). β₂-Tf, together with the typical Tf proteoform in serum, β₁-transferrin (β₁-Tf), were named after their electrophoretic mobility in gel electrophoresis. The conventional method to test for β₂-Tf and β₁-Tf is agarose gel immunofixation electrophoresis (IFE). Although widely used in clinical laboratories, IFE does not provide structural information of the analytes and often leads to ambiguous test results due to the inability to confirm the presence of β₂-Tf. The ambiguity is mainly caused by the existence of additional Tf proteoforms (e.g., additional Tf glycoforms) besides β₁-Tf and β₂-Tf that are present in CSF and other body fluids. During electrophoresis, these additional Tf proteoforms can migrate within or between the β₁-Tf and β₂-Tf band regions, interfering with the detection of β₂-Tf in the IFE test.

In addition, while β₂-Tf is widely used as a diagnostic marker for CSF leak, the primary structure of β₂-Tf as well as that of β₁-Tf has not yet been elucidated.

### SUMMARY

The present disclosure provides methods for analyzing a transferrin glycoform, the methods comprising: (a) capturing a transferrin glycoform on a probe that comprises a transferrin glycoform-binding agent; (b) inserting the probe into the interior capillary of an electrospray emitter; (c) releasing the transferrin glycoform from the probe while it is in the emitter using an elution liquid; (d) nebulizing the transferrin glycoform by electrospray; and (e) analyzing the nebulized transferrin glycoform by mass spectrometry. In some embodiments, the analyzing step of (e) comprises identifying the transferrin glycoform as a β₁-transferrin or a β₂-transferrin glycoform. In some embodiments, the β₁-transferrin glycoform comprises a first and second G2S2 N-glycan. In some cases, the β₁-transferrin glycoform comprises a first G2S2 N-glycan at position 413 and a second G2S2 N-glycan at position 611, according to the amino acid numbering of SEQ ID NO:1. In some embodiments, the β₂-transferrin glycoform comprises a M5 N-glycan and a G0FB N-glycan. In some cases, the β₂-transferrin glycoform comprises a M5 N-glycan at position 413 and a G0FB N-glycan at position 611, according to the amino acid numbering of SEQ ID NO:1.

In some embodiments of said methods for analyzing a transferrin glycoform, the probe is quartz glass. In some embodiments, the probe is a thin-film interferometry (TFI) probe. In some embodiments, said methods further comprise detecting binding of the transferrin glycoform to the binding agent by thin-film interferometry. In some embodiments, said methods further comprise measuring the kinetics of binding of the transferrin glycoform to the binding agent by thin-film interferometry. In some cases, the elution liquid comprises a solvent. In some cases, the elution liquid is delivered by a capillary that is operably connected to capillary of the emitter. In some embodiments, the releasing and nebulizing of the transferrin glycoform are done in the presence of a sheath fluid. In some embodiments of the methods, the mass spectrometry is high-resolution mass spectrometry. In some embodiments of the methods, the analyzing step of (e) is done by a time of flight (TOF), Orbitrap or FT-ICR mass spectrometer.

In some embodiments of the methods for analyzing a transferrin glycoform, the methods further comprise, prior to step (b), dipping the probe into a sample that comprises transferrin glycoforms; washing the probe to remove unbound sample; and detecting binding of transferrin glycoforms to the probe by thin-film interferometry. In some embodiments of the methods, the sample is obtained from a subject suspected of having a CSF leak. In some embodiments, the sample obtained from the subject is a serum sample or a secretion sample. In some cases, the secretion sample is a rhinorrhea or otorrhea secretion sample. In certain embodiments, the sample is a sialic acid depleted sample. In some embodiments of the methods, the transferrin glycoform-binding agent is an antibody. Also provided herein are methods for diagnosing a CSF leak in a subject.

The present disclosure also provides kits for analyzing a transferrin glycoform, said kits comprising (a) a thin-film interferometry (TFI) probe comprising a first binding partner of a binding pair; and (b) a transferrin glycoform-binding agent comprising a second binding partner of a binding pair.

The present disclosure further provides systems comprising an electrospray emitter having an interior capillary and a thin-film interferometry (TFI) probe comprising a transferrin glycoform-binding agent thereon, wherein the probe fits into the interior capillary emitter.

### BRIEF DESCRIPTION OF THE DRAWINGS

FIG. 1A-1D: (A) Experiment workflow of MPIE-ESI-MS for Tf analysis. The MPIE-ESI-MS results of (B) a serum sample. (C) a CSF sample, and (D) a secretion sample from a patient diagnosed of CSF leak: HR-MS raw mass spectra (left) and deconvoluted mass spectra (right) of captured Tf molecules, showing serum-type Tf glycoforms in (B), and both serum-type Tf and brain-type Tf glycoforms in (C) and (D).

FIG. 2A-2B: The MPIE-ESI-MS results of (A) the extract from the gel stripe of the β₁-Tf band region and (B) the extract from the gel stripe of the β₂-Tf band region: HR-MS raw mass spectra (left) and deconvoluted mass spectra (right) of captured Tf molecules, showing β₁-Tf in (A) and β₂-Tf in (B). The image of an agarose gel after gel electrophoresis is posted to the right of the mass spectra, which was placed on the paper template marked with the β₁-Tf and β₂-Tf band regions. The image of a reference agarose gel after immunofixation is posted further on the right, indicating the β₁-Tf and β₂-Tf band regions designated according to the manufacturer's protocol.

FIG. 3A-3B: (A) Primary structure of human transferrin, showing the sequence of 679 amino acids, 19 disulfide bonds, and 2 N-glycosylation sites. (B) N-glycan structures on β₁-Tf (major serum-type Tf) and β₂-Tf (major brain-type Tf), confirmed by comparing the theoretical molecular masses of the Tf glycoforms with the measured molecular masses in FIG. 1, FIG. 2, and Table 1. FIG. 3A: SEQ ID NO: 1.

FIG. 4A-4E: BLI sensorgrams obtained on the 3 BLI microprobes capturing Tf from a serum sample (A), a CSF sample (B), and a secretion sample from a patient diagnosed of CSF leak (C) (same samples as in FIG. 1), and BLI sensorgrams obtained on the 2 BLI microprobes in PBST-B (D) and PBST (E) as negative controls. The BLI signal “jump” between the capture step and the rinse step was caused by refractive index difference between the sample and the buffer (PBST-B for serum samples, PBST for CSF and secretion samples).

FIG. 5A-5C: (A) and (B) The MPIE-ESI-MS results of two CSF samples containing Tf variants: HR-MS raw mass spectra of captured Tf molecules (left) and deconvoluted mass spectra (right), showing the Tf glycoforms. The molecular masses of the putative β₁-Tf and β₂-Tf shifted a same value from those of β₁-Tf and β₂-Tf in normal CSF samples, while the mass difference between the two Tf glycoforms retained as 1546 Da, indicating that the mass shift was caused by amino acid variation in the Tf molecule. (C) The MPIE-ESI-MS result of a CSF sample from an alcohol-consuming patient, showing MS peaks of altered Tf glycoforms that are labeled with presumptive N-glycan structures inferred from the intact molecular masses.

FIG. 6: Shows lectin-agarose mediated depletion of samples can enhance the dynamic range of β₂-Tf detection.

### DEFINITIONS

The terms “polypeptide.” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. See J. Mol. Biol. 48:443-453 (1970).

“Binding” as used herein (e.g. with reference to an anti-transferrin glycoform binding agent, a transferrin-glycoform, and the like) refers to a non-covalent interaction between macromolecules (e.g., between an anti-transferrin glycoform binding agent and a transferrin-glycoform; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}.

The term “glycoform(s)” refers to variants or isoforms of a protein that differ with respect to the number and/or type of glycans (i.e., oligosaccharides) attached to said protein. Typically, varying glycoforms of a protein result from differing post-translational glycosylation of a protein. Glycosylation encompasses a diverse selection of sugar-moiety additions to proteins that ranges from simple monosaccharide modifications to highly complex branched polysaccharide changes. Carbohydrates in the form of asparagine-linked (N-linked) or serine/threonine-linked (O-linked) oligosaccharides are major structural components of many cell surface and secreted proteins.

### DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. As such, the articles “a” and “an” are used herein to refer to one or to more than one (i.e., to at least one) of the grammatical object of the article. By way of example, “an element” means one element or more than one element. Thus, for example, reference to “a cell” includes a plurality of such cells and reference to “the polypeptide” includes reference to one or more polypeptides and equivalents thereof known to those skilled in the art. and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. For example, it is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods for Analyzing Transferrin Glycoforms

The present disclosure provides methods for analyzing a transferrin glycoform, the methods comprising: (a) capturing a transferrin glycoform on a probe that comprises a transferrin glycoform-binding agent; (b) inserting the probe into the interior capillary of an electrospray emitter; (c) releasing the transferrin glycoform from the probe while it is in the emitter using an elution liquid; (d) nebulizing the transferrin glycoform by electrospray; and (e) analyzing the nebulized transferrin glycoform by mass spectrometry.

In some embodiments, the subject methods allow one to directly couple label-free analysis and mass spectrometry (MS) analysis of transferrin glycoforms, particularly between thin-film interferometry (TFI) based label-free analysis and high-resolution MS (HR-MS). This coupling endows the power of MS-based identification of transferrin glycoforms to the label-free characterization of transferrin glycoform biomolecular interactions. A transferrin glycoform is first captured on the surface of a sensing probe during label-free analysis, and then eluted from the probe inside an electrospray emitter and immediately sprayed into a mass spectrometer for HR-MS analysis. HR-MS provides superior resolution and can precisely distinguish and identify different transferrin glycoform variants. The capture of transferrin glycoforms on label-free sensing probes not only allows for real-time monitoring of the binding process through label-free analysis, but also purifies the target transferrin glycoforms to reduce background noise in HR-MS analysis.

Transferrin is a high abundance protein found in vertebrates that plays a role in transporting iron through the body fluids, e.g., plasma. Transferrin is initially translated as a precursor protein which is cleaved to remove its N-terminal signal peptide and post-translationally modified by the asparagine-linked attachment of oligosaccharides, also referred to as N-glycans, to produce a mature transferrin that is secreted into body fluids. N-glycans are typically linked to asparagine residues via an N-acetylglucosamine (GlcNAc) sugar moiety. As mature transferrin glycoproteins typically vary by the structures of the attached N-glycans, these varying transferrin proteoforms are referred to as glycoforms. Several different glycoforms of transferrin exist in humans, containing both sialylated (i.e., comprising sialic acid sugar moieties) and desialylated (i.e., not comprising sialic acid sugar moieties) N-glycans of varying structural configurations.

In some embodiments, the amino acid sequence of a transferrin glycoform that may be analyzed in the subject methods comprises an amino acid sequence having at least 80%, at least 85%, at least 90%, at least 95%, at least 98%, at least 99%, or 100%, amino acid sequence identity to the following human transferrin amino acid sequence:

(SEQ ID NO: 1)
VPDKTVRWCAVSEHEATKCQSFRDHMKSVIPSDGPSVACVKKASYLDCIRAIAANEADAVILDAGLVYDA
YLAPNNLKPVVAEFYGSKEDPQTFYYAVAVVKKDSGFQMNQLRGKKSCHTGLGRSAGWNIPIGLLYCDLP
EPRKPLEKAVANFFSGSCAPCADGTDFPQLCQLCPGCGCSTLNQYFGYSGAFKCLKDGAGDVAFVKHSTI
FENLANKADRDQYELLCLDNTRKPVDEYKDCHLAQVPSHTVVARSMGGKEDLIWELLNQAQEHFGKDKSK
EFQLFSSPHGKDLLFKDSAHGFLKVPPRMDAKMYLGYEYVTAIRNLREGTCPEAPTDECKPVKWCALSHH
ERLKCDEWSVNSVGKIECVSAETTEDCIAKIMNGEADAMSLDGGFVYIAGKCGLVPVLAENYNKSDNCED
TPEAGYFAIAVVKKSASDLTWDNLKGKKSCHTAVGRIAGWNIPMGLLYNKINHCRFDEFFSEGCAPGSKK
DSSLCKLCMGSGLNLCEPNNKEGYYGYTGAFRCLVEKGDVAFVKHQTVPQNTGGKNPDPWAKNLNEKDYE
LLCLDGTRKPVEEYANCHLARAPNHAVVTRKDKEACVHKILRQQQHLFGSNVTDCSGNFCLERSETKDLL
FRDDTVCLAKLHDRNTYEKYLGEEYVKAVGNLRKCSTSSLLEACTFRRP.

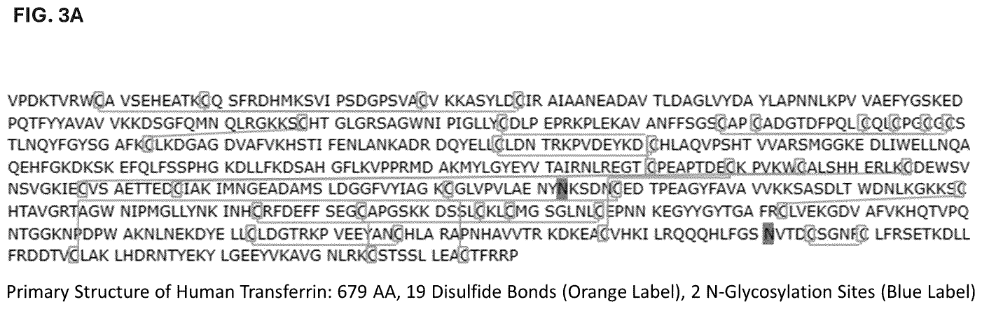

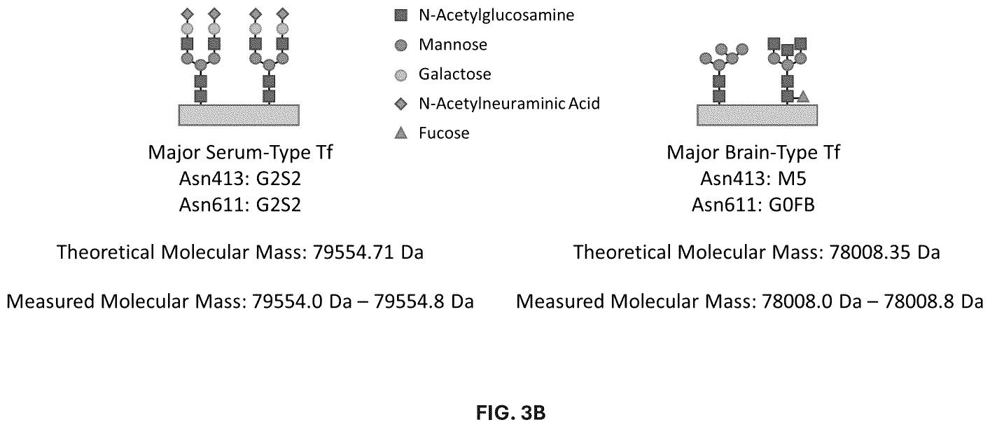

In some embodiments, the amino acid sequence of a transferrin glycoform that may be analyzed in the subject methods comprises an amino acid sequence having at least 80%, at least 85%, at least 90%, at least 95%, at least 98%, at least 99%, or 100%, amino acid sequence depicted in FIG. 3A.

As would be appreciated by the person of skill in the art, the correspondence between the amino acid positions in any given transferrin protein sequence of interest (e.g., a naturally existing protein variant of transferrin) and the amino acid positions of the amino acid sequences depicted in SEQ ID NO:1 or FIG. 3A can be determined by alignment of the respective sequences using known methods in the art.

In some embodiments of the methods for analyzing a transferrin glycoform, the analyzing of the nebulized transferrin glycoform by mass spectrometry in step (e) comprises identifying the transferrin glycoform as a β₁-transferrin glycoform or a β₂-transferrin glycoform. For example, the analyzing may include detecting the mass spectra of the nebulized transferrin glycoform and identifying the transferrin glycoform as a β₁-transferrin or a β₂-transferrin glycoform based on the measured mass spectra.

As defined by the present disclosure, a β₁-transferrin glycoform is characterized by the attachment of a first and second G2S2 N-glycan. A G2S2 N-glycan is a complex-type biantennary N-glycan with terminal sialic acid residues. In some cases, a G2S2 N-glycan has the structure of the G2S2 N-glycan depicted in FIG. 3B. In some cases, a G2S2 N-glycan has a structure defined by Structure 1, below:

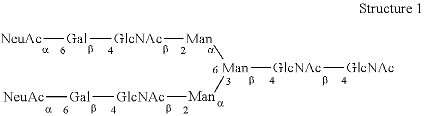

Where: GlcNAc is N-acetylglucosamine; Man is mannose; Gal is galactose; and NeuAc is N-acetylneuraminic acid (i.e., a sialic acid).

In some embodiments, the β₁-transferrin glycoform comprises a G2S2 N-glycan covalently linked to an asparagine (N) residue at a position corresponding to position 413, according to the amino acid numbering of SEQ ID NO:1. In some cases, the β₁-transferrin glycoform comprises a G2S2 N-glycan covalently linked to an asparagine (N) residue at position 413, according to the amino acid numbering of SEQ ID NO:1. In some embodiments, the β₁-transferrin glycoform comprises a G2S2 N-glycan covalently linked to an asparagine (N) residue at a position corresponding to position 611, according to the amino acid numbering of SEQ ID NO:1. In some cases, the β₁-transferrin glycoform comprises a G2S2 N-glycan covalently linked to an asparagine (N) residue at position 611, according to the amino acid numbering of SEQ ID NO:1. In some embodiments, the B1-transferrin glycoform comprises a first G2S2 N-glycan covalently linked to an asparagine (N) residue at a position corresponding to position 413 and a second G2S2 N-glycan covalently linked to an asparagine (N) residue at a position corresponding to position 611, according to the amino acid numbering of SEQ ID NO:1. In a particular embodiment, the β₁-transferrin glycoform comprises a first G2S2 N-glycan covalently linked to an asparagine (N) residue at position 413 and a second G2S2 N-glycan covalently linked to an asparagine (N) residue at position 611, according to the amino acid numbering of SEQ ID NO:1. In certain embodiments, the β₁-transferrin glycoform has a molecular mass of 79554 Da.

As defined by the present disclosure, a β₂-transferrin glycoform is characterized by the attachment of a M5 N-glycan and a G0FB N-glycan. A M5 N-glycan is a desialylated, high mannose-type N-glycan containing unsubstituted terminal mannose sugar moieties. In some cases, the M5 N-glycan has the structure of the M5 N-glycan depicted in FIG. 3B. In some cases, the M5 N-glycan has a structure defined by Structure 2, below:

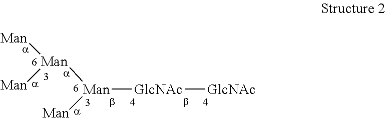

Where: GlcNAc is N-acetylglucosamine; and Man is mannose.

A G0FB glycan is desialylated, biantennary N-glycan with a fucose linked to the first, innermost, GlcNAc and a GlcNAc bisecting the central mannose of the biantennary branch. In some cases, the G0FB N-glycan has the structure of the G0FB N-glycan depicted in FIG. 3B. In some cases, the G0FB N-glycan has a structure defined by Structure 3, below:

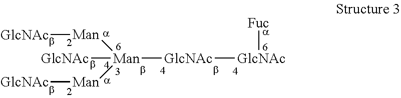

Where: GlcNAc is N-acetylglucosamine; Man is mannose; and Fuc is fucose.

In some embodiments, the β₂-transferrin glycoform comprises a M5 N-glycan covalently linked to an asparagine (N) residue at a position corresponding to position 413, according to the amino acid numbering of SEQ ID NO:1. In some cases, the β₂-transferrin glycoform comprises a M5 N-glycan covalently linked to an asparagine (N) residue at position 413, according to the amino acid numbering of SEQ ID NO:1. In some embodiments, the β₂-transferrin glycoform comprises a G0FB N-glycan covalently linked to an asparagine (N) residue at a position corresponding to position 611, according to the amino acid numbering of SEQ ID NO:1. In some cases, the β₂-transferrin glycoform comprises a G0FB N-glycan covalently linked to an asparagine (N) residue at position 611, according to the amino acid numbering of SEQ ID NO:1. In some embodiments, the β₂-transferrin glycoform comprises a M5 N-glycan covalently linked to an asparagine (N) residue at a position corresponding to position 413 and a G0FB N-glycan covalently linked to an asparagine (N) residue at a position corresponding to position 611, according to the amino acid numbering of SEQ ID NO:1. In a particular embodiment, the β₂-transferrin glycoform comprises a M5 N-glycan covalently linked to an asparagine (N) residue at position 413 and a G0FB N-glycan covalently linked to an asparagine (N) residue at position 611, according to the amino acid numbering of SEQ ID NO:1. In certain embodiments, the β₂-transferrin glycoform has a molecular mass of 78008 Da.

In some embodiments, the identifying the transferrin glycoform as a β₁-transferrin or a β₂-transferrin glycoform may be accomplished by measuring the expected difference in molecular mass between the β₁-transferrin and β₂-transferrin glycoforms (e.g., in the cases where a transferrin protein variant resulting from a genetic polymorphism is being analyzed) In such cases, while the exact molecular mass of each gycoform may differ due to the variant protein sequence, the difference in molecular mass between the β₁-transferrin and β₂-transferrin glycoforms does not. In some cases, the identifying the transferrin glycoform as a β₁-transferrin or a β₂-transferrin glycoform comprises measuring a molecular mass difference of 1546 Da between the respective glycoforms, where the β₂-transferrin glycoform has a molecular mass that is 1546 Da less than the molecular mass of the β₁-transferrin glycoform.

To implement the subject methods, a transferrin glycoform is first captured on the surface of a probe comprising a transferrin glycoform-binding agent. The probe may be composed of any suitable solid substrate such as, e.g., glass, quartz glass, or plastic (e.g., polylactic acid, polyethylene, polypropylene, and polytetrafluouroethylene plastics). Different immobilization chemistries may be used to covalently link transferrin glycoform-binding agents to the surface of the probe. By way of example, bifunctional reagents containing a siloxane group for chemical conjugation to quartz (i.e., SiO₂) and a hydroxyl, amine, carboxyl or other suitable reaction groups may be used for the attachment of biological molecules such as proteins (e.g., antibodies, antigens, streptavidin). As another example, a variety of chemical conjugation methods are available with polymers (e.g., plastics) which utilize available chemically-active surface groups, such as amine, hydroxyl, and carboxyl groups.

In some embodiments, the probe may be pre-coated with conjugated streptavidin, which enables subsequent coating of the probe with a suitable transferrin glycoform-binding agent that is, itself, conjugated to biotin (e.g., an anti-transferrin antibody conjugated to biotin). For example, in such cases, the probe may be coated with a suitable transferrin glycoform-binding agent just prior to its use in the subject methods.

In some embodiments, the probe is a thin-film interferometry (TFI) probe. In these embodiments, the methods may further comprise detecting binding of the transferrin glycoform to the binding agent by thin-film interferometry. In some cases, the methods may further comprise measuring the kinetics of binding of the transferrin glycoform to the binding agent by thin-film interferometry. TFI probes generally contain a quartz glass rod having a diameter of less than 1 mm having specialized optical layers and specialized surface chemistry built at the distal end (the sensing end) of the probe. Thin-film interferometry (TFI) probes, systems containing the same and methods for their use are described in, e.g., US20110305599A1, Luo et al (Clin Chim Acta 2020 502:128-132), Luo et al (Clinical Infectious Diseases 2021 73: e3095-e3097) and Luo et al (Clin Chem 2020 66:1319-1328), which are incorporated by reference herein for disclosure of the probes, systems containing the same and methods. Briefly, a TFI probe employs the phenomenon of thin-film spectral interference to detect the binding of an analyte of interest. A thin-film layer comprising molecules that bind to an analyte of interest is bonded to the sensing end of the probe. The interface of the quartz glass rod bonded to the thin-film layer forms a first reflecting surface (i.e., an internal reference layer) while the molecules that bind to the analyte of interest (i.e., the thin film layer) form a second reflecting surface. Critically, the refractive index of the thin-film layer differs from the refractive index of the quartz glass rod. White light from a light source is guided through the quartz glass rod and may be reflected back to a detector (e.g., a spectrophotometer) from the first reflecting surface or may travel through to the thin-film layer and be reflected back to the detector from the second reflecting surface. Due to differing refractive indices of the quartz glass rod and thin-film layer, a phase-shift occurs between the light waves reflected from first and second reflecting surfaces which leads constructive or destructive interference of the light waves. The degree of phase-shift is dependent on the path length of the light waves through the thin-film layer. When analytes of interest bind and accumulate in the thin-film layer, the path length of the light waves through the thin-film layer (and thus the degree of phase-shift) is altered, leading to detectable change in the interference pattern of the reflected light waves. Shifts in interference due to the accumulation of biomolecules are monitored in real-time to sensitively analyze and calculate rates of association and dissociation among target proteins with high precision.

Following capture of the transferrin glycoform on the probe, the probe is inserted into the interior capillary of an electrospray emitter. Electrospray emitters, systems containing the same, and methods for their use are described in, e.g., Olshina, Maya A., and Michal Sharon. “Mass spectrometry: a technique of many faces.” Quarterly reviews of biophysics 49 (2016): e18 and Domon, Bruno, and Ruedi Aebersold. “Mass spectrometry and protein analysis.” science 312.5771 (2006): 212-217, which are incorporated by reference herein for disclosure of the electrospray emitters, systems containing the same and methods.

The methods further comprise releasing the transferrin glycoform from the probe while it is in the emitter using an elution liquid and nebulizing the transferrin glycoform by electrospray. In some embodiments, the elution liquid comprises a solvent suitable for eluting the transferrin-glycoform and compatible with the electrospray emitter and the mass spectrometer. In some cases, the solvent is a combination of acetonitrile and formic acid in aqueous solution (e.g., a solution of 80% acetonitrile and 2% formic acid). In some embodiments, the elution liquid is delivered by a capillary that is operably connected to capillary of the emitter. The electrospray emitter may be filled with a suitable sheath fluid and the releasing and nebulizing of the transferrin-glycoform is done in the presence of a sheath fluid. Suitable sheath fluids include, without limitation, ammonium formate (e.g., 10 mM ammonium formate).

Suitable methods for inserting probes with captured analytes into the interior capillary of an electrospray emitter; releasing the analytes from the probe while it is in the emitter using an elution liquid; and nebulizing the analytes by electrospray for spraying into a mass spectrometer are also described in US20230305019A and Luo, Ruben Yiqi, and Samuel Yang. “Microprobe-capture in-emitter elution: An affinity capture technique to directly couple a label-free optical sensing technology with mass spectrometry for protein analysis.” Analytical Chemistry 95.13 (2023): 5494-5499, the disclosures of which are incorporated by reference herein.

The analyzing of the nebulized transferrin glycoform is performed by mass spectrometry. Suitable mass spectrometers for use in the subject methods include, without limitation, quadrupole, time-of-flight (TOF), Fourier transform ion cyclotrons (FT-IC) and ion traps, such as the Orbitrap. Mass spectrometers, systems containing the same, and methods for their use are described in, e.g., in Olshina, Maya A., and Michal Sharon. “Mass spectrometry: a technique of many faces.” Quarterly reviews of biophysics 49 (2016): e18 and Domon, Bruno, and Ruedi Aebersold. “Mass spectrometry and protein analysis.” science 312.5771 (2006): 212-217, which are incorporated by reference herein for disclosure of the mass spectrometers, systems containing the same and methods. In some embodiments, the analyzing of the nebulized transferrin glycoform is performed by high-resolution mass spectrometry (HR-MS). High-resolution mass spectrometry systems and methods are described, e.g., in Lai, Yin-Hung, and Yi-Sheng Wang. “Advances in high-resolution mass spectrometry techniques for analysis of high mass-to-charge ions.” Mass Spectrometry Reviews 42.6 (2023): 2426-2445, the disclosure of which is incorporated by reference herein.

As discussed above, a subject probe of the present disclosure comprises a transferrin glycoform-binding agent. In some embodiments, a suitable transferrin glycoform-binding agent is an anti-transferrin antibody. Exemplary anti-transferrin antibodies include, without limitation, MAB5746 from R&D Systems, AHP858 from Bio-Rad Laboratories, A80-128A from Bethyl Laboratories, Inc., MMO1 or MM06 from Sinobiological, and T2027 from MilliporeSigma.

In certain embodiments, the methods for analyzing a transferrin glycoform, further comprise, prior to step (b), dipping the probe into a sample that comprises transferrin glycoforms; washing the probe to remove unbound sample; and detecting binding of transferrin glycoforms to the probe by thin-film interferometry.

In some embodiments, the sample is a sample obtained from a subject suspected of having a cerebrospinal fluid (CSF) leak. CSF leaks can be caused by trauma (e.g., craniofacial trauma), as a side effect of various surgical interventions, or sometimes spontaneously.

Symptoms associated with CSF leaks can include low-pressure (orthostatic) headaches, nausea, neck pain, loss of smell or taste, and ringing in the ear. Thus, in some cases, a subject which has experienced trauma and/or displays one or more of the symptoms above may be suspected of having a CSF leak. In some embodiments the subject is a vertebrate. In some embodiments, the subject is a mammal. In some embodiments, the subject is a human.

The sample obtained from the subject suspected of having a CSF leak may be drawn from any suitable body fluid (e.g., any body fluid other than the cerebrospinal fluid). In certain embodiments, the sample obtained from the subject suspected of having a CSF leak is a serum sample. In some embodiments, the sample obtained from the subject suspected of having a CSF leak is a secretion sample. Secretion samples may be any sample of body fluid that is secreted from a bodily orifice. In some cases, the secretion is a rhinorrhea secretion (i.e. a nose secretion) sample. In some cases, the secretion is an otorrhea secretion (i.e., car secretion) sample. In some embodiments, the sample obtained from the subject may diluted in water or an appropriate buffer known in the art (e.g., a phosphate-buffered saline buffer) prior to dipping the subject probe into the sample.

In certain embodiments, prior to dipping the subject probe into the sample, the sample may be depleted for sialic acids (i.e., depleted for any molecules containing sialic acid moieties such as sialylated transferrin glycoforms). By way of example, where increased sensitivity of detection for the β₂-transferrin glycoform is desired, the sample may be depleted of β₁-transferrin glycoforms, as well as any other sialylated transferrin glycoforms. For example, the β₁-transferrin glycoform is highly abundant in serum and thus depletion of sialylated transferrin glycoforms in a rhinorrhea or otorrhea sample contaminated with serum (e.g., due to bleeding) may be performed to reduce background levels of the β₁-transferrin glycoform in the sample and increase the sensitivity of detection for the β₂-transferrin glycoform. A sample may be depleted of sialic acids using a sialic acid-binding agent. In some embodiments, the sialic acid binding agent is a lectin. The lectin may, by way of example, be conjugated to an agarose substrate (e.g., agarose beads) which the sample is passed over, thus capturing and removing any sialic acids in the sample to produce a sialic acid depleted sample. Lectin conjugated agarose beads are known in the art and readily commercially available.

Following dipping of the probe into the sample, the probe may be washed one or more times in an appropriate buffer to remove unbound sample (e.g., any compounds or molecules in the sample that have not been captured by the transferrin glycoform-binding agents of the probe). The appropriate buffer may be chosen by the skilled person based on their knowledge in the art. In some cases, the washing buffer is a phosphate-buffered saline (PBS) buffer, optionally comprising a detergent (e.g. tween).

Following washing of the probe to remove unbound sample, binding of transferrin glycoforms to the probe is detected by thin-film interferometry (e.g., in embodiments of the subject methods where the probe is a thin-film interferometry probe). As discussed above, thin-film interferometry (TFI) probes, systems containing the same and methods for their use are described in, e.g., US20110305599A1, Luo et al (Clin Chim Acta 2020 502:128-132), Luo et al (Clinical Infectious Diseases 2021 73: e3095-e3097) and Luo et al (Clin Chem 2020 66:1319-1328), which are incorporated by reference herein for disclosure of the probes, systems containing the same and methods.

### Methods for Diagnosing Cerebrospinal Fluid (CSF) Leaks in a Subject

The present disclosure provides methods for diagnosing a CSF leak in a subject, the methods comprising: (a) dipping a probe into a sample obtained from a subject suspected of having a CSF leak, wherein the probe comprises a transferrin glycoform-binding agent; (b) capturing a transferrin glycoform present in the sample on the probe; (c) releasing the transferrin glycoform from the probe while it is in the emitter using an elution liquid; (d) nebulizing the transferrin glycoform by electrospray; and (e) analyzing the nebulized transferrin glycoform by mass spectrometry, wherein the diagnosing of a CSF leak in the subject is based on the analysis of the transferrin glycoforms in the sample from the subject. Steps (a)-(e) of the subject methods for diagnosing a CSF leak in a subject may be carried out according to the subject methods of analyzing a transferrin glycoform extensively described herein.

CSF leaks occur when there is tear or perforation in the outermost layer of the meninges that protects the central nervous system. CSF leaks can be caused by trauma (e.g., craniofacial trauma), as a side effect of various surgical interventions, or sometimes spontaneously. Symptoms associated with CSF leaks can include low-pressure (orthostatic) headaches, nausea, neck pain, loss of smell or taste, and ringing in the ear. Thus, in some cases, a subject which has experienced trauma and/or displays one or more of the symptoms above may be suspected of having a CSF leak.

In some embodiments, the subject may be a vertebrate (e.g., fish, amphibian, reptile, bird, mammal), a mammal (e.g., an ungulate (e.g., a pig, a cow, a goat, a sheep); a rodent (e.g., a rat, a mouse); a non-human primate; a human; a feline (e.g., a cat); a canine (e.g., a dog); etc.), and the like. In some embodiments the subject is a human.

The sample obtained from the subject suspected of having a CSF leak may be drawn from any suitable body fluid (e.g., any body fluid other than the cerebrospinal fluid). In certain embodiments, the sample obtained from the subject suspected of having a CSF leak is a serum sample. In some embodiments, the sample obtained from the subject suspected of having a CSF leak is a secretion sample. Secretion samples may be any sample of body fluid that is secreted from a bodily orifice. In some cases, the secretion is a rhinorrhea secretion (i.e. a nose secretion) sample. In some cases, the secretion is an otorrhea secretion (i.e., car secretion) sample.

In some embodiments, the sample obtained from the subject may diluted in water or an appropriate buffer known in the art (e.g., a phosphate-buffered saline buffer) prior to dipping the subject probe into the sample. In certain embodiments, prior to dipping the subject probe into the sample, the sample may be depleted for sialic acids (i.e., depleted for any molecules containing sialic acid moieties such as sialylated transferrin glycoforms). By way of example, where increased sensitivity of detection for the β₂-transferrin glycoform is desired, the sample may be depleted of β₁-transferrin glycoforms, as well as any other sialylated transferrin glycoforms. For example, the β₁-transferrin glycoform is highly abundant in serum and thus depletion of sialylated transferrin glycoforms in a rhinorrhea or otorrhea sample contaminated with serum (e.g., due to bleeding) may be performed to reduce background levels of the β₁-transferrin glycoform in the sample and increase the sensitivity of detection for the β₂-transferrin glycoform. A sample may be depleted of sialic acids using a sialic acid-binding agent. In some embodiments, the sialic acid binding agent is a lectin. The lectin may, by way of example, be conjugated to an agarose substrate (e.g., agarose beads) which the sample is passed over, thus capturing and removing any sialic acids in the sample to produce a sialic acid depleted sample. Lectin conjugated agarose beads are known in the art and readily commercially available.

Following dipping of the probe into the sample, the probe may be washed one or more times in an appropriate buffer to remove unbound sample (e.g., any compounds or molecules in the sample that have not been captured by the transferrin glycoform-binding agents of the probe). The appropriate buffer may be chosen by the skilled person based on their knowledge in the art. In some cases, the washing buffer is a phosphate-buffered saline (PBS) buffer, optionally comprising a detergent (e.g. tween).

In some embodiments, the diagnosing of a CSF leak in the subject comprises detection of the β₂-transferrin glycoform, as defined herein, in the sample obtained from the subject (i.e., a sample from a body fluid other than CSF). In certain embodiments, the identifying the transferrin glycoform as a β₂-transferrin glycoform may be accomplished by measuring the expected difference in molecular mass between the β₁-transferrin and β₂-transferrin glycoforms (e.g., in the cases where a subject may have a transferrin protein variant resulting from a genetic polymorphism) In such cases, while the exact molecular mass of each gycoform may differ due to the variant protein sequence, the difference in molecular mass between the β₁-transferrin and β₂-transferrin glycoforms does not. In some cases, the identifying the transferrin glycoform as a β₂-transferrin glycoform comprises measuring a molecular mass difference of 1546 Da between β₁-transferrin and β₂-transferrin glycoforms, where the β₂-transferrin glycoform has a molecular mass that is 1546 Da less than the molecular mass of the β₁-transferrin glycoform.

### Kits

The present disclosure provides kits which may be used for carrying out the subject methods described herein, the kits comprising: (a) a thin-film interferometry (TFI) probe comprising a first binding partner of a binding pair; and (b) a transferrin glycoform-binding agent comprising a second binding partner of a binding pair.

The first and second binding partners of a subject binding pair may be chosen such that the probe may be prepared for use by coating the probe with the transferrin glycoform-binding agent, where the transferrin glycoform-binding agent will be captured by the probe via the binding of the first and second binding partners of the binding pair. The first and second binding partners of the binding pair may be any suitable molecules of a binding pair (e.g., a ligand and receptor pair, an antibody and antigen pair, a protein and small molecule binding pair, etc.) In some cases, the binding pair is a avidin or streptavidin protein and a biotin molecule. In these embodiments, the first binding agent may be avidin or streptavidin and the second binding agent may be biotin. In some cases, the first binding agent may be biotin and the second binding agent may be a avidin or streptavidin.

TFI probes generally contain a quartz glass rod having a diameter of less than 1 mm having specialized optical layers and specialized surface chemistry built at the distal end (the sensing end) of the probe. Thin-film interferometry (TFI) probes, systems containing the same and methods for their use are described in, e.g., US 20110305599A1, Luo et al (Clin Chim Acta 2020 502:128-132), Luo et al (Clinical Infectious Diseases 2021 73: e3095-e3097) and Luo et al (Clin Chem 2020 66:1319-1328), which are incorporated by reference herein for disclosure of the probes, systems containing the same and methods. Briefly, a TFI probe employs the phenomenon of thin-film spectral interference to detect the binding of an analyte of interest. A thin-film layer comprising molecules that bind to an analyte of interest is bonded to the sensing end of the probe. The interface of the quartz glass rod bonded to the thin-film layer forms a first reflecting surface (i.e., an internal reference layer) while the molecules that bind to the analyte of interest (i.e., the thin film layer) form a second reflecting surface. Critically, the refractive index of the thin-film layer differs from the refractive index of the quartz glass rod. White light from a light source is guided through the quartz glass rod and may be reflected back to a detector (e.g., a spectrophotometer) from the first reflecting surface or may travel through to the thin-film layer and be reflected back to the detector from the second reflecting surface. Due to differing refractive indices of the quartz glass rod and thin-film layer, a phase-shift occurs between the light waves reflected from first and second reflecting surfaces which leads constructive or destructive interference of the light waves. The degree of phase-shift is dependent on the path length of the light waves through the thin-film layer. When analytes of interest bind and accumulate in the thin-film layer, the path length of the light waves through the thin-film layer (and thus the degree of phase-shift) is altered, leading to detectable change in the interference pattern of the reflected light waves. Shifts in interference due to the accumulation of biomolecules are monitored in real-time to sensitively analyze and calculate rates of association and dissociation among target proteins with high precision.

Different immobilization chemistries may be used to covalently link the surface of the probe to transferrin glycoform-binding agents. By way of example, bifunctional reagents containing a siloxane group for chemical conjugation to quartz (i.e., SiO₂) and a hydroxyl, amine, carboxyl or other suitable reaction groups may be used for the attachment of if biological molecules such as proteins (e.g., antibodies, antigens, streptavidin). As another example, a variety of chemical conjugation methods are available with polymer (e.g., plastics) which utilize available chemically-active surface groups, such as amine, hydroxyl, and carboxyl groups. In some embodiments, the probe may be pre-coated with conjugated streptavidin, which enables subsequent coating with a suitable transferrin glycoform-binding agent that is, itself, conjugated to biotin (e.g., an anti-transferrin antibody conjugated to biotin).

In some embodiments, a suitable transferrin glycoform-binding agent is an anti-transferrin antibody. Exemplary anti-transferrin antibodies include, without limitation, MAB5746 from R&D Systems, AHP858 from Bio-Rad Laboratories, A80-128A from Bethyl Laboratories, Inc., MMO1 or MM06 from Sinobiological, and T2027 from MilliporeSigma. These antibodies may be conjugated to the second binding partner of a binding pair using known methods in the art. For example, in some cases, the antibodies are conjugated to biotin, the conjugation performed using known methods in the art.

A subject kit may further comprise one or more containers for storing the TFI probe and the transferrin glycoform-binding agent. In some cases, the size of the container may depend on the volume of the transferrin glycoform-binding agent (in lyophilized or liquid form) to be held in the container. In certain embodiments, the container may be configured to hold an amount of a subject transferrin glycoform-binding agent, ranging from 0.1 mg to 1000 mg, such as from 0.1 mg to 900 mg, such as from 0.1 mg to 800 mg, such as from 0.1 mg to 700 mg, such as from 0.1 mg to 600 mg, such as from 0.1 mg to 500 mg, such as from 0.1 mg to 400 mg, or 0.1 mg to 300 mg, or 0.1 mg to 200 mg, or 0.1 mg to 100 mg, 0.1 mg to 90 mg, or 0.1 mg to 80 mg, or 0.1 mg to 70 mg, or 0.1 mg to 60 mg, or 0.1 mg to 50 mg, or 0.1 mg to 40 mg, or 0.1 mg to 30 mg, or 0.1 mg to 25 mg, or 0.1 mg to 20 mg, or 0.1 mg to 15 mg, or 0.1 mg to 10 mg, or 0.1 mg to 5 mg, or 0.1 mg to 1 mg, or 0.1 mg to 0.5 mg. In some cases, the container is configured to hold an amount of transferrin glycoform-binding agent (in lyophilized or liquid form), ranging from 0.1 g to 10 g, or 0.1 g to 5 g, or 0.1 g to 1 g, or 0.1 g to 0.5 g. In some instances, the container may be configured to hold a volume (e.g., a volume of a liquid) ranging from 0.1 ml to 1000 ml, such as from 0.1 ml to 900 ml, or 0.1 ml to 800 ml, or 0.1 ml to 700 ml, or 0.1 ml to 600 ml, or 0.1 ml to 500 ml, or 0.1 ml to 400 ml, or 0.1 ml to 300 ml, or 0.1 ml to 200 ml, or 0.1 ml to 100 ml, or 0.1 ml to 50 ml, or 0.1 ml to 25 ml, or 0.1 ml to 10 ml, or 0.1 ml to 5 ml, or 0.1 ml to 1 ml, or 0.1 ml to 0.5 ml. In some cases, the container is configured to hold a volume ranging from 0.1 ml to 200 ml.

Suitable containers for the compositions include, for example, boxes, bottles, vials, syringes, and test tubes. Containers can be formed from a variety of suitable materials, including glass or plastic. For example, the container may be composed of glass, such as, but not limited to, silicate glass, borosilicate glass, sodium borosilicate glass (e.g., PYREX™), fused quartz glass, fused silica glass, and the like. Other examples of suitable materials for the containers include plastics, such as, but not limited to, polypropylene, polymethylpentene, polytetrafluoroethylene (PTFE), perfluoroethers (PFE), fluorinated ethylene propylene (FEP), perfluoroalkoxy alkanes (PFA), polyethylene terephthalate (PET), polyethylene (PE), polyetheretherketone (PEEK), and the like.

In some embodiments, the container may be sealed. That is, the container may include a seal that substantially prevents the contents of the container from exiting the container. The seal of the container may also substantially prevent other substances from entering the container. For example, the seal may be a water-tight seal that substantially prevents liquids from entering or exiting the container, or may be an air-tight seal that substantially prevents gases from entering or exiting the container. In some instances, the seal is a removable or breakable seal, such that the contents of the container may be exposed to the surrounding environment when so desired, e.g., if it is desired to remove a portion of the contents of the container. In some instances, the seal is made of a resilient material to provide a barrier (e.g., a water-tight and/or air-tight seal) for retaining a sample in the container. Particular types of seals include, but are not limited to, films, such as polymer films, caps, etc., depending on the type of container. Suitable materials for the seal include, for example, rubber or polymer seals, such as, but not limited to, silicone rubber, natural rubber, styrene butadiene rubber, ethylene-propylene copolymers, polychloroprene, polyacrylate, polybutadiene, polyurethane, styrene butadiene, and the like, and combinations thereof. A container may have a sterile access port (for example, the container may be an a vial having a stopper pierceable by a hypodermic injection needle).

### Systems

The present disclosure provides systems that may, e.g., be used to analyze transferrin glycoforms according to the subject methods. Said systems comprise: an electrospray emitter having an interior capillary; and a thin-film interferometry (TFI) probe comprising a transferrin glycoform-binding agent thereon, wherein the probe fits into the interior capillary of the emitter.

Electrospray emitters having interior capillaries, systems containing the same, and methods for their use are described in, e.g., Olshina, Maya A., and Michal Sharon. “Mass spectrometry: a technique of many faces.” Quarterly reviews of biophysics 49 (2016): e18 and Domon, Bruno, and Ruedi Aebersold. “Mass spectrometry and protein analysis.” science 312.5771 (2006): 212-217, which are incorporated by reference herein for disclosure of the electrospray emitters, systems containing the same and methods.

Suitable electrospray emitters are further described in, e.g., US20230305019A and Luo, Ruben Yiqi, and Samuel Yang. “Microprobe-capture in-emitter elution: An affinity capture technique to directly couple a label-free optical sensing technology with mass spectrometry for protein analysis.” Analytical Chemistry 95.13 (2023): 5494-5499, the disclosures of which are incorporated by reference herein.

Thin-film interferometry (TFI) probes, systems containing the same and methods for their use are described in, e.g., US20110305599A1, Luo et al (Clin Chim Acta 2020 502:128-132), Luo et al (Clinical Infectious Diseases 2021 73: e3095-e3097) and Luo et al (Clin Chem 2020 66:1319-1328), which are incorporated by reference herein for disclosure of the probes, systems containing the same and methods. Different immobilization chemistries may be used to covalently link the surface of the probe to transferrin glycoform-binding agents. By way of example, bifunctional reagents containing a siloxane group for chemical conjugation to quartz (i.e., SiO₂) and a hydroxyl, amine, carboxyl or other suitable reaction groups may be used for the attachment of if biological molecules such as proteins (e.g., antibodies, antigens, streptavidin). As another example, a variety of chemical conjugation methods are available with polymers (e.g., plastics) which utilize available chemically-active surface groups, such as amine, hydroxyl, and carboxyl groups. The size and shape of the probe may be chosen as desired to fit into the interior capillary of a corresponding electrospray emitter, such that the probe can be inserted into the interior capillary of the subject emitter to enable release and nebulization of transferrin glycoforms captured on the subject probe.

As discussed above, the TFI probe of a subject system comprises a transferrin glycoform-binding agent. In some embodiments, a suitable transferrin glycoform-binding agent is an anti-transferrin antibody. Exemplary anti-transferrin antibodies include, without limitation, MAB5746 from R&D Systems, AHP858 from Bio-Rad Laboratories, A80-128A from Bethyl Laboratories, Inc., MMO1 or MM06 from Sinobiological, and T2027 from MilliporeSigma.

In some embodiments of the subject systems, the emitter is operably connected to a reservoir of elution liquid. The reservoir may be operably connected to the emitter via a liquid capillary. In some cases, the reservoir of elution liquid and the emitter may be operably connected to a fluid pump, such that the fluid pump is configured to pump liquid from the reservoir into the emitter. The fluid pump may be any suitable fluid pump known in the art. For example, in some cases, the fluid pump may be a pneumatic pump. In some embodiments, the elution liquid comprises a solvent suitable for eluting the transferrin-glycoform and compatible with the electrospray emitter and the mass spectrometer. In some cases, the solvent is a combination of acetonitrile and formic acid in aqueous solution (e.g., a solution of 80% acetonitrile and 2% formic acid).

In some embodiments, a subject system further comprises a mass spectrometer. Suitable mass spectrometers for use in the subject systems include, without limitation, quadrupole, time-of-flight (TOF), Fourier transform ion cyclotrons (FT-IC) and ion traps, such as the Orbitrap. Mass spectrometers, systems containing the same, and methods for their use are described in, e.g., in Olshina, Maya A., and Michal Sharon. “Mass spectrometry: a technique of many faces.” Quarterly reviews of biophysics 49 (2016): e18 and Domon, Bruno, and Ruedi Aebersold. “Mass spectrometry and protein analysis.” science 312.5771 (2006): 212-217, which are incorporated by reference herein for disclosure of the mass spectrometers, systems containing the same and methods.

In some embodiments, a subject system further comprises a spectrophotometer. The spectrophotometer may be operably connected to the TFI probe to detect changes in the interference pattern of white light reflected from the distal end (the sensing end) of the probe. The TFI probe in operable connection to the spectrophotometer enables label-free TFI analysis of transferrin glycoforms captured on the probe. Spectrophotometers and their use in TFI systems are described, e.g., in US20110305599A1, Luo et al (Clin Chim Acta 2020 502:128-132), Luo et al (Clinical Infectious Diseases 2021 73: e3095-e3097) and Luo et al (Clin Chem 2020 66:1319-1328), which are incorporated by reference herein for disclosure of the spectrophotometers, systems containing the same, and methods.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt. nucleotide(s); i.m., intramuscular(ly); i.p., intraperitoneal(ly); s.c., subcutaneous(ly); and the like.

### Example 1: Study of β1-Transferrin and β2-Transferrin Using Microprobe-Capture In-Emitter Elution and High-Resolution Mass Spectrometry

### Introduction

There has been extensive basic research on human Tf since it is a high-abundance protein in blood with a major role in iron metabolism. The amino acid sequence of Tf precursor was determined through protein cleavage and cDNA characterization, showing a full sequence of 698 amino acids; after the removal of an N-terminal signal peptide, the mature form of Tf contains 679 amino acids, 19 intramolecular disulfide bonds formed between cysteine residues, and two N-glycans attached to the amino groups of the side chains of Asn419 and Asn611 (Asn432 and Asn630 of Tf precursor). Tf proteoforms typically vary by the N-glycan structures, and the Tf proteoforms of interest so far are Tf glycoforms, including β₁-Tf and β₂-Tf.

Although it is known that β₂-Tf has desialylated N-glycans while β₁-Tf has fully sialylated N-glycans, the primary structures of β₁-Tf and β₂-Tf have not been clarified. On the other hand, the N-glycans on Tf glycoforms in serum and in CSF were characterized using gel electrophoresis, liquid chromatography, and enzymatic digestion-based mass spectrometry (MS) by neurobiologists. It was reported that a group of Tf glycoforms were present in serum, namely serum-type Tf glycoforms or sTf. among which a specific Tf glycoform (major serum-type Tf) predominated; the serum-type Tf glycoforms also existed in CSF. An additional group of Tf glycoforms were present in CSF, namely brain-type Tf glycoforms, among which a specific Tf glycoform (major brain-type Tf) was more abundant than the rest. The brain-type Tf glycoforms was at least partly synthesized in the CSF-producing tissue choroid plexus rather than being produced by glycosidase digestion of serum-type Tf glycoforms. It was found that the N-glycans on the major serum-type Tf consisted of bi-antennary oligosaccharide chains with sialylated terminals, and those on the brain-type Tf glycoforms were desialylated, or more specifically, unsialylated (asialotransferrin). It was hypothesized that the major brain-type Tf was β₂-Tf, but this hypothesis has not been proved. Similarly, it is reasonable to presume that the major serum-type Tf was β₁-Tf, however a proof is required.

In clinical cases where CSF leak is suspected, secretion samples can be collected from patients and sent to clinical laboratories to test for the presence of β₂-Tf. The conventional method to test β₂-Tf as well as β₁-Tf is agarose gel immunofixation electrophoresis (IFE). Although it is widely used in clinical laboratories, it does not provide structural information of the analytes. Thus, the primary structures of β₁-Tf and β₂-Tf, particularly the N-glycan structures on these Tf glycoforms, remained an unanswered question with this method.

As an emerging technology in clinical diagnostics, high-resolution mass spectrometry (HR-MS), particularly top-down HR-MS, can be used to analyze a protein target in its intact state and identify the post-translational modifications and amino acid variations in its proteoforms. While HR-MS is an ideal tool to study the Tf glycoforms, the quality of data acquired during HR-MS analysis depends on sample preparation. In this article, an innovative affinity capture technique for sample preparation, called microprobe-capture in-emitter elution (MPIE), was incorporated with HR-MS to study the Tf glycoforms. MPIE can directly couple a label-free optical sensing technology with MS. The label-free optical sensing technology is next-generation biolayer interferometry (BLI, also named as thin-layer interferometry, TFI), which senses optical thickness changes on the sensing surface of a microprobe caused by biomolecular interactions such as antibody-antigen binding, achieving real-time measurement without employing a reporter molecule (enzyme, fluorophore, etc.). To implement MPIE, an analyte is first captured on the surface of a microprobe, and subsequently eluted from the microprobe inside an electrospray emitter. The capture process is monitored in real-time via BLI. When electrospray is established from the emitter to a mass spectrometer, the analyte is immediately ionized via electrospray ionization (ESI) for HR-MS analysis. By this means, BLI and HR-MS are directly coupled in the form of MPIE-ESI-MS, which is readily deployed to study the Tf glycoforms and the primary structures of β₁-Tf and β₂-Tf.

### Methods

### Materials and Specimens

LC-MS grade water, acetonitrile, formic acid, and 0.2 μm PVDF syringe filters were purchased from Thermo Fisher Scientific (Waltham, MA). A mouse monoclonal anti-transferrin IgG antibody (anti-Tf Ab) was obtained from Sinobiological (Wayne, PA), and biotinylated using an EZ-Link HPDP-Biotin reagent kit (Waltham, MA). Remnant CSF and serum samples from patients, and secretion samples from patients suspected of CSF leak were obtained from Stanford Health Care and Stanford Children's Health, following an approved institutional review board protocol for the use of remnant patient specimens.

### Sample Preparation

The biotinylated anti-Tf Ab was diluted in phosphate-buffered saline at pH 7.4 with 0.02% Tween 20 and 0.2% BSA (PBST-B) to 10 μg/ml for use. A pooled CSF sample was made by mixing 9 CSF samples from patients to explore the analytical sensitivity of MPIE-ESI-MS for β₂-Tf in CSF. The pooled CSF sample was mixed with water to make a dilution series. CSF samples were 1:1 diluted in phosphate-buffered saline at pH 7.4 with 0.02% Tween 20 (PBST) and serum samples were 1:19 diluted in PBST-B. Secretion samples from patients were first mixed with an equal amount of water and filtered using a 0.2 μm PVDF syringe filter, and then 1:1 diluted in PBST.

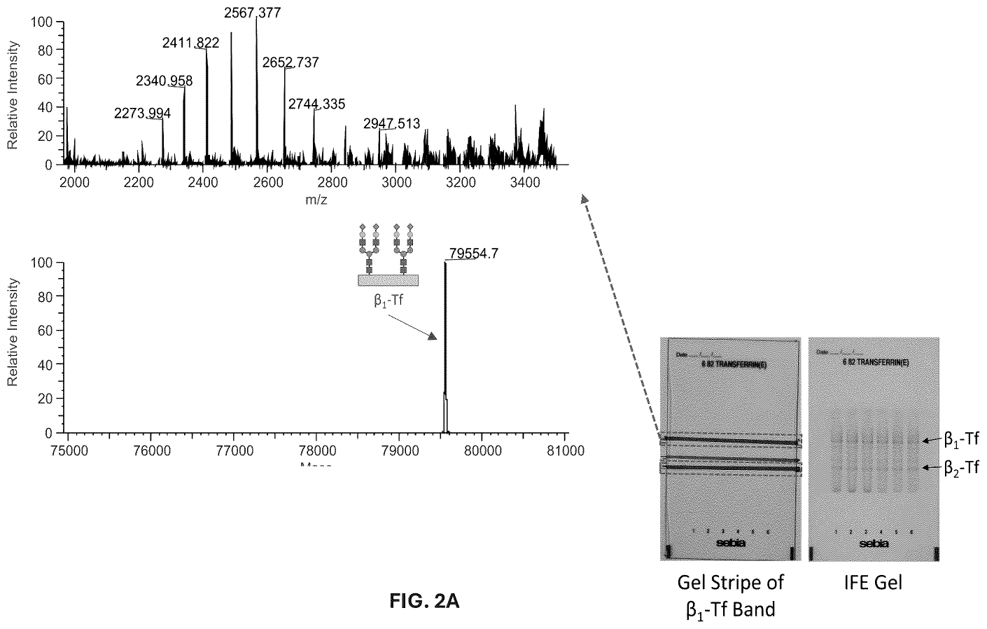

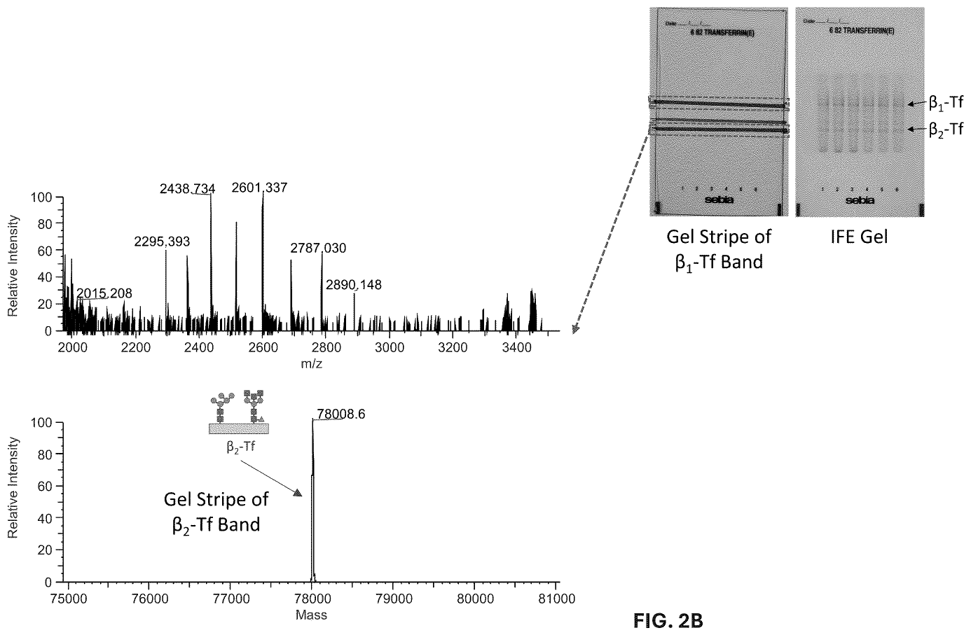

To study the gel electrophoresis-separated Tf glycoforms, gel electrophoresis of CSF samples was carried out using a Hydragel 6 β₂ Transferrin kit (Sebia, Lisses, France) following the manufacturer's protocol. In brief, a CSF sample was first 1:1 mixed with an iron-saturating solution, and then 10 μl of the sample was loaded to each of the 6 wells on an agarose gel so that replicates were run in all the 6 lanes. Gel electrophoresis was implemented in a Hydrasys 2 instrument (Sebia, Lisses, France), and the agarose gel was removed from the instrument immediately after the gel electrophoresis was completed, without carrying out the immunofixation steps. The agarose gel was placed on a paper template marked with the β₁-Tf and β₂-Tf band regions, and the gel stripes of the band regions were cut out using a scalpel. Each gel stripe was placed in a 1.5 ml sample tube, 200 μl PBST was added, and the sample was rocked for 2 hr at room temperature to extract the analyte from the gel stripe. After extraction, the supernatant was filtered using a 0.2 μm PVDF syringe filter. The images of an agarose gel for MPIE-ESI-MS analysis and a reference agarose gel after immunofixation are posted in FIG. 2A and 2B.

### MPIE-ESI-MS Instrumentation and Experiment

An MPIE-ESI-MS experiment consists of two parts: BLI-based affinity capture and in-emitter elution ESI-MS, with the details including instrumentation described elsewhere. The BLI-based affinity capture was implemented in a Gator Plus analyzer (Gator Bio, Palo Alto, CA): a BLI microprobe pre-coated with streptavidin was first dipped into the biotinylated anti-Tf Ab solution for 10 min to load the anti-Tf Ab, then dipped into a sample for 10 min to capture Tf molecules, and rinsed in PBST for 1 min to remove non-specifically bound molecules. The in-emitter elution ESI-MS was implemented in an EMASS-II ESI ion source which coupled an ECE-001 capillary electrophoresis instrument (CMP Scientific, Brooklyn, NY) with an Orbitrap Q-Exactive Plus mass spectrometer (Thermo Scientific, San Jose, CA): an electrospray emitter with a regular open end and a tapered open end (tip orifice diameter 20-30 μm) was filled with a sheath liquid (10 mM ammonium formate in water); after affinity capture, the microprobe was rinsed in the sheath liquid for 10 s, inserted into the emitter through the regular open end, and settled in the tapered end by gravity; the emitter was mounted to the ESI ion source, and a capillary was inserted into the emitter through the regular open end and positioned right behind the microprobe to deliver an elution liquid (80% acetonitrile and 2% formic acid in water); once electrospray was established by applying a positive voltage to the sheath liquid in the emitter, HR-MS data acquisition was initiated, and injection of the elution liquid was started subsequently. The emitter was placed ˜2 mm away from the mass spectrometer inlet with the electrospray voltage set at 2.2 kV. The injection of the elution liquid was driven by 5 psi pneumatic pressure. The following MS parameters were used: ion-transfer capillary temperature 350° C., S-lens RF level 50, and number of microscans 10. Primary mass spectra were acquired in positive polarity at resolution 17.5K.

### Data Analysis

In HR-MS analysis of proteins, it is necessary to deconvolute raw MS data to merge the multiple charge states and isotopic peaks of an analyte to obtain its accurate molecular mass. The acquired data in each MPIE-ESI-MS experiment was viewed as a time trace of MS responses, and the elution time window of an analyte was identified by checking the molecular ions of the analyte at each time point. The data in the elution time window were selected for deconvolution using Biopharma Finder 4.1 (Thermo Fisher Scientific, San Jose, CA) with the ReSpect algorithm. MS peaks of analytes were displayed in deconvoluted mass spectra at uncharged state showing average molecular masses.

### Results

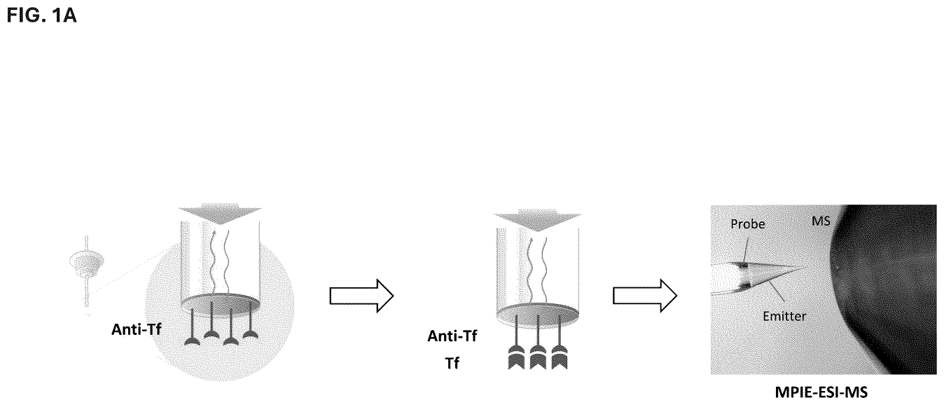

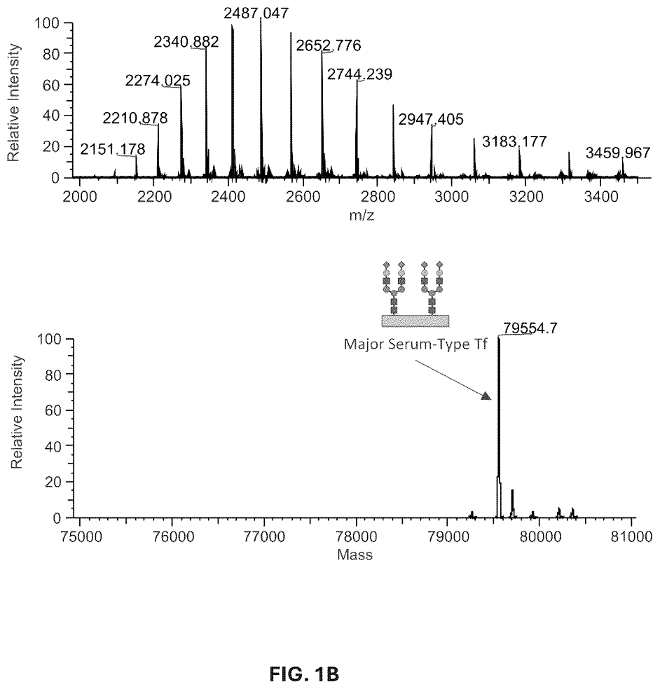

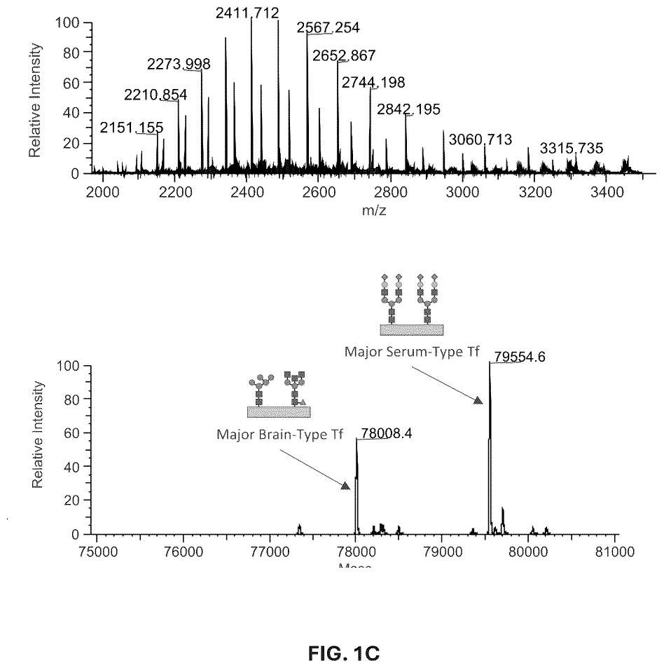

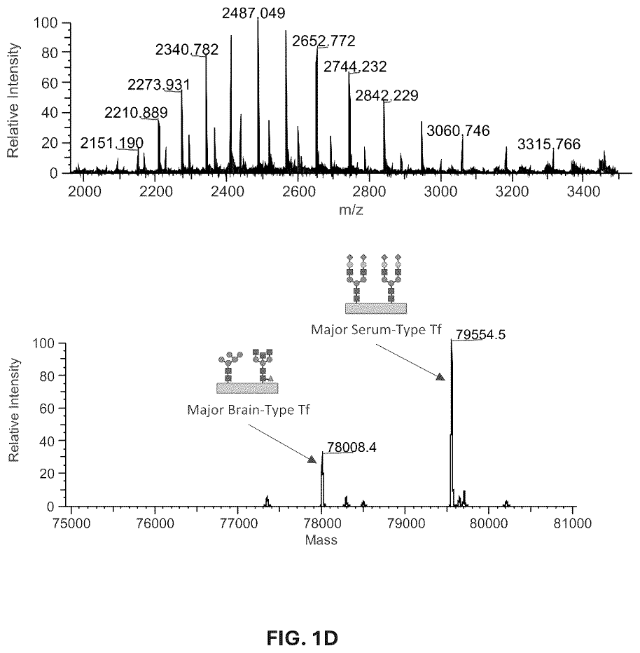

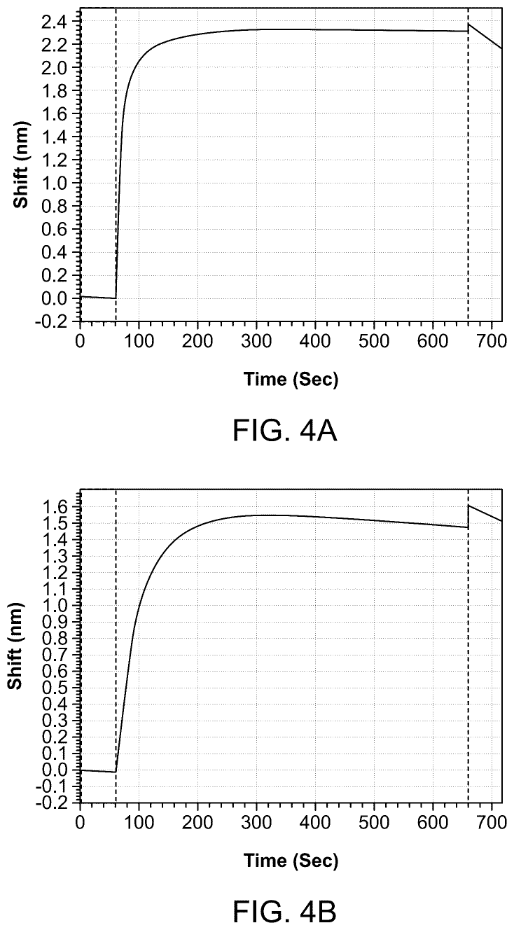

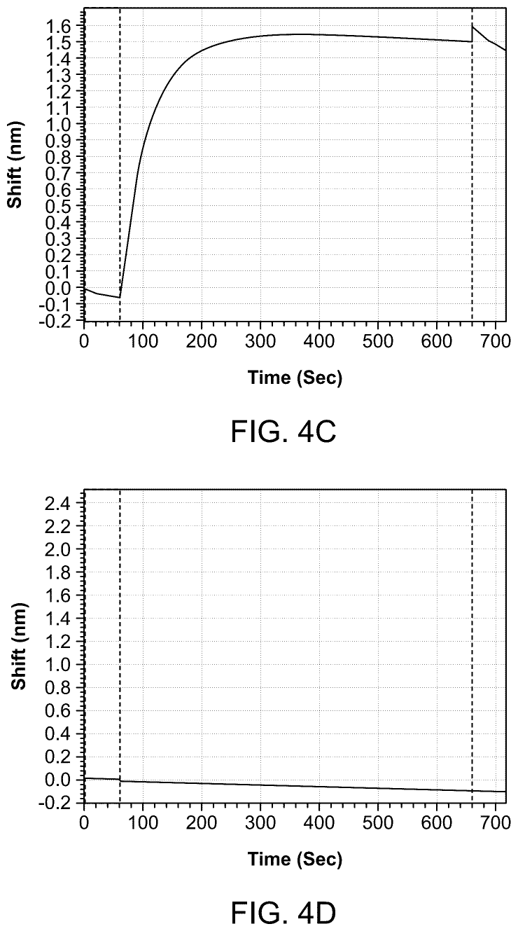

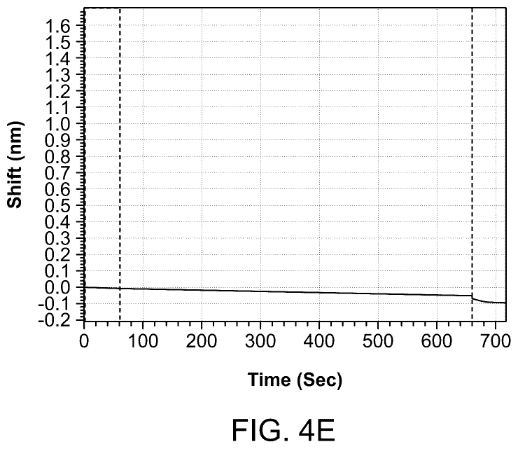

The performance of MPIE-ESI-MS for Tf analysis was demonstrated and reported elsewhere. MPIE-ESI-MS had a limit of detection for the Tf standard at 0.063 μg/ml, which was translated to no more than 7 fmol Tf molecules captured on a microprobe. The high analytical sensitivity and specificity provided by affinity capture made MPIE-ESI-MS an ideal method to study the Tf molecules. The results of a set of serum, CSF, and secretion samples are shown in FIGS. 1A-1D. The deconvoluted mass spectrum of the serum sample showed a group of MS peaks around 79554 Da; they are mainly serum-type Tf glycoforms and the predominant MS peak at 79554 Da is the major serum-type Tf (N-glycan structures shown in FIGS. 1A-1D, see details in Discussion). The deconvoluted mass spectrum of the CSF sample showed the major serum-type Tf and a group of MS peaks around 78008 Da; they are mainly brain-type Tf glycoforms and the most abundant MS peak at 78008 Da is the major brain-type Tf (N-glycan structures shown in FIGS. 1A-1D, see details in Discussion). The deconvoluted mass spectrum of the secretion sample showed both serum-type and brain-type Tf glycoforms, meaning that CSF was present in the sample. This finding was consistent with the fact that the secretion sample was obtained from a patient diagnosed with CSF leak. In addition, the BLI sensorgrams (a time trace of label-free optical sensing responses) during the capture of Tf as well as the BLI sensorgrams of negative controls (PBST-B and PBST) are shown in FIGS. 4A-4E. The BLI measurement facilitates real-time monitoring of the capture process.

To figure out the relationship between the major serum-type Tf, the major brain-type Tf and β₁-Tf, β₂-Tf, after gel electrophoresis of CSF samples, the extracts from the gel stripes of the β₁-Tf and β₂-Tf band regions were analyzed using MPIE-ESI-MS. The results of a CSF sample are shown in FIGS. 2A-2B. The deconvoluted mass spectra showed only the MS peak of the major serum-type Tf in the extract from the β₁-Tf band region and only the MS peak of the major brain-type Tf in the extract from the β₂-Tf band region. The measured accurate molecular masses were consistent with the experiments in FIGS. 1A-1D and matched the theoretical molecular masses of the major serum-type Tf and major brain-type Tf (N-glycan structures shown in FIGS. 2A-2B, see details in Discussion). This observation proved the hypothesis that β₁-Tf and β₂-Tf were actually the major serum-type Tf and major brain-type Tf, respectively. In addition, the gel area between the β₁-Tf and β₂-Tf band regions was also analyzed and minor Tf glycoforms in serum and CSF were not found, which could be due to the low quantities of the minor Tf glycoforms in a gel stripe.

A collection of 11 secretion samples from patients suspected of CSF leak were analyzed using the MPIE-ESI-MS method, among which 5 samples were positive for β₂-Tf and the rest were negative as measured by the conventional IFE test. As shown in Table 1, the MS peak at 78008 Da was observed in the MPIE-ESI-MS results of the 5 positive samples but not found in those of the 6 negative samples, which confirmed the consistency between the MPIE-ESI-MS method and the conventional IFE test. In addition, the limit of detection of the MPIE-ESI-MS method was explored. A pooled CSF sample was mixed with water at 1:1, 1:4, 1:9, and 1:19 ratios to prepare a dilution series for analysis. The peak intensities decreased with the pooled CSF sample dilution, and it was demonstrated that the MPIE-ESI-MS method was able to detect β₂-Tf in at least 10-fold diluted CSF (1:9 pooled CSF: water mixture) (data not shown).

TABLE 1
The MPIE-ESI-MS results of 11 secretion samples
from patients suspected of CSF leak.

| | β1-Tf | | β2-Tf | |
|---|---|---|---|---|
| | | Measured | | Measured |
| | | Molecular | | Molecular |
| Secretion Sample | Detected? | Mass | Detected? | Mass |
| Positive Sample 1 | Yes | 79554.50 Da | Yes | 78008.42 Da |
| Positive Sample 2 | Yes | 79554.79 Da | Yes | 78008.60 Da |
| Positive Sample 3 | Yes | 79554.75 Da | Yes | 78008.83 Da |
| Positive Sample 4 | Yes | 79554.04 Da | Yes | 78008.68 Da |
| Positive Sample 5 | Yes | 79554.77 Da | Yes | 78008.03 Da |
| Negative Sample 1 | Yes | 79554.29 Da | No | / |
| Negative Sample 2 | Yes | 79554.80 Da | No | / |
| Negative Sample 3 | Yes | 79554.81 Da | No | / |
| Negative Sample 4 | No | / | No | / |
| Negative Sample 5 | Yes | 79554.49 Da | No | / |
| Negative Sample 6 | Yes | 79554.73 Da | No | / |

### Discussion

The MPIE-ESI-MS analysis of the extracts from the gel stripes proved that β₁-Tf and β₂-Tf were identical to the major serum-type Tf and the major brain-type Tf, respectively. As Tf glycoforms, β₁-Tf and β₂-Tf share the amino acid backbone but contain varying N-glycans. The amino acid sequence of Tf was previously investigated and reported. The primary structure of a mature Tf molecule is composed of 679 amino acids with 19 disulfide bonds and 2 N-glycans, as illustrated in FIG. 3A. N-glycans on proteins typically include bi-, tri-, or tetra-antennary oligosaccharide chains resulted from sequential action of glycosyltransferases, and an N-glycan can be named according to its sugar composition and branching structure (traditional nomenclature used in this article). When the potential varieties of N-glycans are established, the structures of Tf glycoforms can be confirmed by aligning the measured accurate molecular masses with the theoretical molecular masses of the potential Tf glycoforms.

The potential structures of N-glycans on Tf were previously reported by employing enzymatic digestion-based MS analysis of the Tf glycoforms in CSF. It was revealed that the most abundant N-glycan type was G2S2. Quantitation of the digested glycopeptides encompassing the glycosylation sites found that G2S2 consisted of roughly 65% of all N-glycans on Asn413 and 73% of all N-glycans on Asn611. Thus, the Tf glycoform with two G2S2 N-glycans on Asn413 and Asn611 aligns with β₁-Tf for the following reasons: (1) the measured molecular mass of β₁-Tf exactly matches its theoretical molecular mass 79554.71 Da (FIG. 3B), (2) it is the most abundant Tf glycoform in serum and CSF, and (3) it is consistent with the fact that β₁-Tf is fully sialylated (N-acetylneuraminic acid as the sialic acid). In addition, the Tf glycoforms associated with M5 and G0FB N-glycans must be the most abundant unsialylated Tf glycoforms in CSF provided that the two N-glycans were found to be the most abundant unsialylated N-glycans. Particularly, M5 was found consisting of roughly 20% of all N-glycans on Asn413 and G0FB was found consisting of 26% of all N-glycans on Asn611. Among the possible combinations of the two N-glycans, the Tf glycoform with an M5 N-glycan and a G0FB N-glycan matches β₂-Tf because (1) the measured molecular mass of β₂-Tf exactly matches its theoretical molecular mass 78008.35 Da (FIG. 3B), and (2) it is consistent with the fact that β₂-Tf is unsialylated. The glycosylation sites of the M5 and G0FB N-glycans were found to be on Asn413 and Asn611 respectively by another study of digested glycopeptides from Tf glycoforms in CSF. The presence of a bisecting N-acetylglucosamine (GlcNAc) in the G0FB N-glycan was proved by the previously reported lectin-binding experiments, verifying the structure of G0FB N-glycan over another possible isomeric N-glycan without a bisecting GlcNAc. Thus, the analysis above substantiates the following findings: (1) β₁-Tf, the major serum-type Tf, has two G2S2 N-glycans on Asn413 and Asn611; and (2) β₂-Tf, the major brain-type Tf, has an M5 N-glycan on Asn413 and a G0FB N-glycan on Asn611.

Besides the two major Tf glycoforms in CSF (β₁-Tf and β₂-Tf), it is known that other minor Tf glycoforms may also exist in CSF, blood, and other body fluids. As illustrated in the previous literature, there are a variety of unsialylated N-glycans on Tf molecules, and the sialylated N-glycan G2S2 also has diversity such as the more branched form G3S3. Thus, a number of fully sialylated, partially sialylated, and unsialylated Tf glycoforms can be formed by the various combinations of the two N-glycans on a Tf molecule. In gel electrophoresis, sialic acids bring negative charges to a Tf molecule under neutral or alkaline pH conditions, influencing its electrophoretic mobility. As such, Tf glycoforms migrate in the order of fully sialylated, disialylated, and unsialylated Tf glycoforms, with regard to the number of sialic acids on the N-glycans. Thus, the minor Tf glycoforms can migrate within or between the β₁-Tf and β₂-Tf band regions. For instance, the product insert of the Hydragel 6 β₂ Transferrin kit states that a band of disialylated Tf glycoforms (disialotransferrin) may exist above the β₂-Tf band. On the other hand, the unsialylated Tf glycoforms besides β₂-Tf can migrate within the β₂-Tf band region and interfere with the β₂-Tf detection. The product insert suggests to use the ratio of unsialylated to disialylated bands to confirm the presence of β₂-Tf. The reason of this practice can be explained as follows: when CSF is present in a sample, the abundance of the major unsialylated Tf glycoform β₂-Tf significantly exceeds the minor unsialylated and disialylated Tf glycoforms, resulting in a high ratio of the β₂-Tf band to the disialotransferrin band. This practice in the IFE test can be achieved in the MPIE-ESI-MS analysis as all the Tf glycoforms are shown in a deconvoluted mass spectrum and the MS peak intensities represent their relative quantities. However, it is probably unnecessary because β₂-Tf can be specifically detected by its accurate molecular mass and definitively differentiated from other unsialylated and disialylated Tf glycoforms.

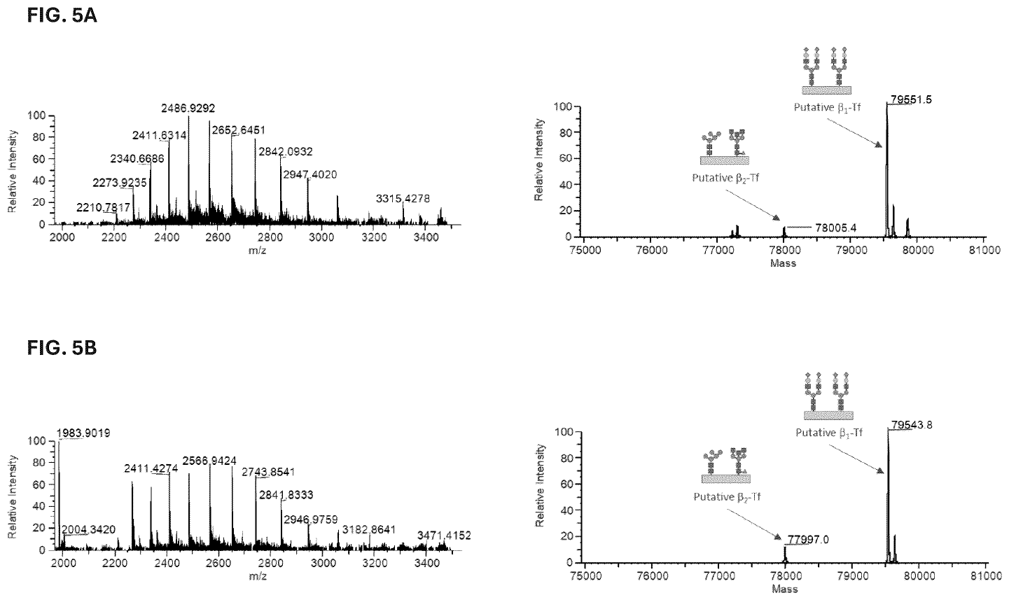

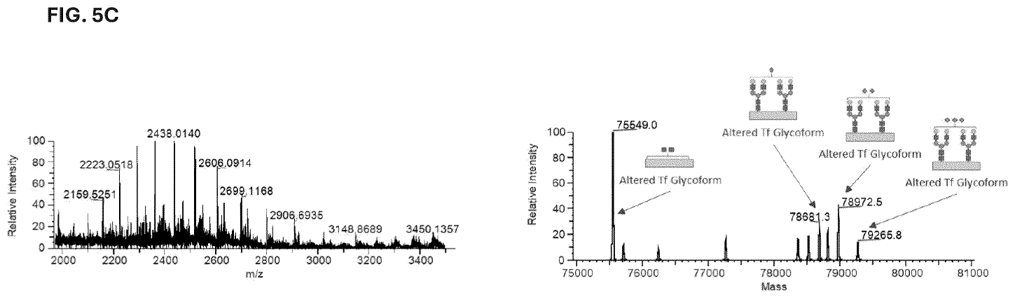

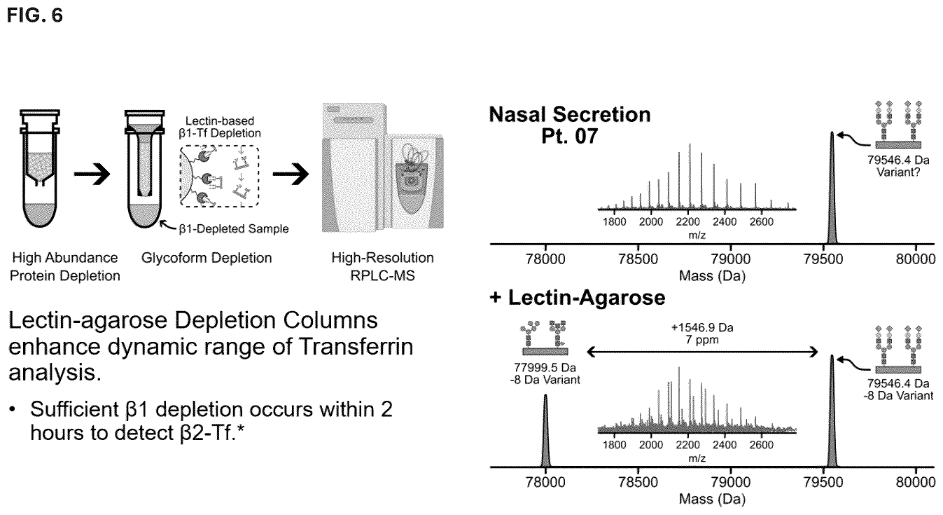

The demonstration of MPIE-ESI-MS in detection of β₂-Tf paved a way to establish an MS-based clinical assay for β₂-Tf. When implementing the accurate molecular mass-based detection of β₂-Tf, Tf variants resulted from genetic polymorphism should be taken into consideration. In theory, amino acid variation in the Tf molecule can change the molecular masses of β₁-Tf and β₂-Tf but not the molecular mass difference between the two Tf glycoforms (1546 Da), provided the two glycosylation sites are not modified. This hypothesis is supported by the MPIE-ESI-MS results of a few Tf variant-containing CSF samples, as shown in FIG. 5A and 5B. In addition, the N-glycans on serum-type Tf glycoforms can vary under specific pathophysiological conditions such as carbohydrate-deficient syndromes and alcohol abuse. β₁-Tf might not be found in those samples due to aberrant glycosylation. As an example, FIG. 5C shows altered Tf glycoforms in a CSF sample from an alcohol-consuming patient.

As an innovative affinity capture technique, MPIE facilitates real-time monitoring of the affinity capture process to overcome the lack of process monitoring in conventional affinity capture techniques. The assembly of a BLI microprobe and an electrospray emitter restricts dispersion of eluted analyte in MPIE-ESI-MS and substantially brings up the concentration of the minute amount of analyte captured by a BLI microprobe, allowing for good analytical sensitivity. Currently MPIE-ESI-MS is not an ideal tool for quantitative analysis due to the variation in capture agent loading and analyte binding on a BLI microprobe, as well as that in MS ionization. In the future, when accurate quantitation is needed, quantitative MPIE-ESI-MS analysis should be achievable by employing internal standards, i.e., preferably stable isotope-labeled analytes.

### References

1. Warnecke A, Averbeck T, Wurster U, Harmening M, Lenarz T, Stöver T. Diagnostic Relevance of β2-Transferrin for the Detection of Cerebrospinal Fluid Fistulas. Arch Otolaryngol Neck Surg. 2004;130(10):1178. doi: 10.1001/archotol.130.10.1178
2. McCudden C R, Senior B A, Hainsworth S, et al. Evaluation of high resolution gel β₂-transferrin for detection of cerebrospinal fluid leak. Clin Chem Lab Med CCLM. 2013;51(2):311-315. doi:10.1515/cclm-2012-0408
3. Papadea C, Schlosser R J. Rapid Method for β2-Transferrin in Cerebrospinal Fluid Leakage Using an Automated Immunofixation Electrophoresis System. Clin Chem. 2005;51(2):464-470. doi:10.1373/clinchem.2004.042697
4. Görögh T, Rudolph P, Meyer J E, Werner J A, Lippert B M, Maune S. Separation of β2-Transferrin by Denaturing Gel Electrophoresis to Detect Cerebrospinal Fluid in Ear and Nasal Fluids. Clin Chem. 2005;51(9):1704-1710. doi:10.1373/clinchem.2005.054916
5. Zaret D L, Morrison N, Gulbranson R, Keren D F. Immunofixation to Quantify β2-Transferrin in Cerebrospinal Fluid to Detect Leakage of Cerebrospinal Fluid from Skull Injury. Clin Chem. 1992;38(9):1909-1912. doi:10.1093/clinchem/38.9.1909
6. Nandapalan V, Watson I D, Swift A C. Beta-2-transferrin and cerebrospinal fluid rhinorrhoea. Clin Otolaryngol. 1996;21(3):259-264. doi:10.1111/j.1365-2273.1996.tb01737.x
7. Gallo P, Bracco F, Morara S, Battistin L, Tavolato B. The cerebrospinal fluid transferrin/Tau proteins. J Neurol Sci. 1985;70(1):81-92. doi:10.1016/0022-510X(85)90190-X
8. Yang F, Lum J B, McGill J R, et al. Human transferrin: cDNA characterization and chromosomal localization. Proc Natl Acad Sci. 1984;81(9):2752-2756. doi:10.1073/pnas.81.9.2752
9. MacGillivray R T, Mendez E, Sinha S K, Sutton M R, Lineback-Zins J, Brew K. The complete amino acid sequence of human serum transferrin. Proc Natl Acad Sci. 1982;79(8):2504-2508. doi:10.1073/pnas.79.8.2504
10. Wang S, Kaltashov I A. Identification of Reduction-Susceptible Disulfide Bonds in Transferrin by Differential Alkylation Using O ¹⁶/O ¹⁸ Labeled Iodoacetic Acid. J Am Soc Mass Spectrom. 2015;26(5):800-807. doi:10.1007/s13361-015-1082-5
11. de Jong G, van Dijk J P, van Eijk H G. The biology of transferrin. Clin Chim Acta. 1990;190(1-2):1-46. doi:10.1016/0009-8981(90)90278-Z
12. de Jong G, van Eijk H G. Microheterogeneity of human serum transferrin: A biological phenomenon studied by isoelectric focusing in immobilized pH gradients. Electrophoresis. 1988;9(9):589-598. doi:10.1002/elps.1150090921
13. de Jong G, van Noort W L, van Eijk H G. Carbohydrate analysis of transferrin subfractions isolated by preparative isoelectric focusing in immobilized pH gradients. Electrophoresis. 1992;13(1):225-228. doi:10.1002/elps.1150130146
14. Delaroche O, Bordureb P, Lippert E, Sagnieza M. Perilymph detection by β2-transferrin immunoblotting assay. Application to the diagnosis of perilymphatic fistulae. Clin Chim Acta. 1996;245(1):93-104. doi:10.1016/0009-8981(95)06177-0
15. Hoffmann A, Nimtz M, Getzlaff R, Conradt H S. ‘Brain-type’ N-glycosylation of asialo-transferrin from human cerebrospinal fluid. FEBS Lett. 1995;359(2-3):164-168. doi:10.1016/0014-5793(95)00034-7
16. Futakawa S, Nara K, Miyajima M, et al. A unique N-glycan on human transferrin in CSF: a possible biomarker for iNPH. Neurobiol Aging. 2012;33(8):1807-1815. doi:10.1016/j.neurobiolaging.2011.02.023
17. Brown K J, Vanderver A, Hoffman E P, Schiffmann R, Hathout Y. Characterization of transferrin glycopeptide structures in human cerebrospinal fluid. Int J Mass Spectrom. 2012;312:97-106. doi:10.1016/j.ijms.2011.06.021
18. Nagae M, Morita-Matsumoto K, Arai S, et al. Structural change of N-glycan exposes hydrophobic surface of human transferrin. Glycobiology. 2014;24(8):693-702. doi:10.1093/glycob/cwu033
19. Hoshi K, Ito H, Abe E, et al. Transferrin Biosynthesized in the Brain Is a Novel Biomarker for Alzheimer's Disease. Metabolites. 2021;11(9):616. doi:10.3390/metabo11090616
20. Hoshi K, Matsumoto Y, Ito H, et al. A unique glycan-isoform of transferrin in cerebrospinal fluid: A potential diagnostic marker for neurological diseases. Biochim Biophys Acta BBA-Gen Subj. 2017;1861(10):2473-2478. doi:10.1016/j.bbagen.2017.07.005
21. Caslavska J, Schild C, Thormann W. High-resolution capillary zone electrophoresis and mass spectrometry for distinction of undersialylated and hypoglycosylated transferrin glycoforms in body fluids. J Sep Sci. 2020;43(1):241-257. doi:10.1002/jssc.201900857
22. Siuti N, Kelleher N L. Decoding protein modifications using top-down mass spectrometry. Nat Methods. 2007;4(10):817-821. doi:10.1038/nmeth 1097
23. Brown K A, Melby J A, Roberts D S, Ge Y. Top-down proteomics: challenges, innovations, and applications in basic and clinical research. Expert Rev Proteomics. 2020;17(10):719-733. doi:10.1080/14789450.2020.1855982
24. Luo R Y, Wong C, Xia J Q, Glader B E, Shi R Z, Zehnder J L. Neutral-Coating Capillary Electrophoresis Coupled with High-Resolution Mass Spectrometry for Top-Down Identification of Hemoglobin Variants. Clin Chem. Published online Oct. 29, 2022:hvac171. doi:10.1093/clinchem/hvac171
25. Donnelly D P, Rawlins C M, DeHart C J, et al. Best practices and benchmarks for intact protein analysis for top-down mass spectrometry. Nat Methods. 2019;16(7):587-594. doi:10.1038/s41592-019-0457-0
26. Padula M, Berry I, O'Rourke M, Raymond B, Santos J, Djordjevic S P. A Comprehensive Guide for Performing Sample Preparation and Top-Down Protein Analysis. Proteomes. 2017;5(4):11. doi:10.3390/proteomes5020011
27. Luo R Y, Yang S. Microprobe-Capture In-Emitter Elution: An Affinity Capture Technique to Directly Couple a Label-Free Optical Sensing Technology with Mass Spectrometry for Protein Analysis. Anal Chem. 2023;95(13):5494-5499. doi:10.1021/acs.analchem.2c04727
28. Luo Y R, Chakraborty I, Lazar-Molnar E, Wu AHB, Lynch K L. Development of Label-Free Immunoassays as Novel Solutions for the Measurement of Monoclonal Antibody Drugs and Antidrug Antibodies. Clin Chem. 2020;66(10):1319-1328. doi:10.1093/clinchem/hvaa179
29. Luo Y R, Yun C, Chakraborty I, Wu AHB, Lynch K L. A SARS-CoV-2 Label-Free Surrogate Virus Neutralization Test and a Longitudinal Study of Antibody Characteristics in COVID-19 Patients. Tang Y W, ed. J Clin Microbiol. 2021;59(7). doi:10.1128/JCM.00193-21
30. Bieberich E. Synthesis, Processing, and Function of N-glycans in N-glycoproteins. In: Yu R K, Schengrund C L, eds. Glycobiology of the Nervous System. Vol 9. Advances in Neurobiology. Springer New York; 2014:47-70. doi:10.1007/978-1-4939-1154-7_3
31. Cao L, Diedrich J K, Ma Y, et al. Global site-specific analysis of glycoprotein N-glycan processing. Nat Protoc. 2018;13(6):1196-1212. doi:10.1038/nprot.2018.024
32. Maier M, Reusch D, Bruggink C, Bulau P, Wuhrer M, Mølhøj M. Applying mini-bore HPAEC-MS/MS for the characterization and quantification of Fc N-glycans from heterogencously glycosylated IgGs. J Chromatogr B. 2016;1033-1034:342-352. doi:10.1016/j.jchromb.2016.08.001
33. Kleinert P, Kuster T, Durka S, et al. Mass Spectrometric Analysis of Human Transferrin in Different Body Fluids. Clin Chem Lab Med. 2003;41(12). doi:10.1515/CCLM.2003.241
34. Coddeville B, Carchon H, Jacken J, Briand G, Spik G. Determination of glycan structures and molecular masses of the glycovariants of serum transferrin from a patient with carbohydrate deficient syndrome type II. Glycoconj J. 1998;15(3):265-273. doi:10.1023/A:1006997012617
35. HYDRAGEL 6 ß2 TRANSFERRIN(E) Kit Instruction Manual. Published online March 2011.
36. Caslavska J, Lanz C, Burda P, Tobler M, Thormann W. Analysis of genetic variants of transferrin in human serum after desialylation by capillary zone electrophoresis and capillary isoelectric focusing. J Sep Sci.2017;40(11):2488-2497. doi:10.1002/jssc.201700211
37. Lee P L, Halloran C, Trevino R, Felitti V, Beutler E. Human transferrin G277S mutation: a risk factor for iron deficiency anaemia: Transferrin G277S Mutation and Anaemia. Br J Haematol. 2001;115(2):329-333. doi:10.1046/j.1365-2141.2001.03096.x
38. Pang H, Koda Y, Soejima M, Kimura H. Identification of a mutation (A1879G) of transferrin from cDNA prepared from peripheral blood cells. Ann Hum Genet. 1998;62(3):271-274. doi:10.1046/j.1469-1809.1998.6230271.x
39. Evans R W, Crawley J B, Garratt R C, et al. Characterization and Structural Analysis of a Functional Human Serum Transferrin Variant and Implications for Receptor Recognition. Biochemistry. 1994;33(41):12512-12520. doi:10.1021/bi00207a019
40. Arndt T. Carbohydrate-deficient transferrin as a marker of chronic alcohol abuse: a critical review of preanalysis, analysis, and interpretation. Clin Chem. 2001;47(1):13-27.
41. Helander A, Eriksson G, Stibler H, Jeppsson J O. Interference of transferrin isoform types with carbohydrate-deficient transferrin quantification in the identification of alcohol abuse. Clin Chem. 2001;47(7):1225-1233.
42. Helander A, Wielders J, Anton R, et al. Reprint of Standardisation and use of the alcohol biomarker carbohydrate-deficient transferrin (CDT). Clin Chim Acta. 2017;467:15-20. doi:10.1016/j.cca.2017.03.018

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1. A method for analyzing a transferrin glycoform, comprising:
(a) capturing a transferrin glycoform on a probe that comprises a transferrin glycoform-binding agent;
(b) inserting the probe into the interior capillary of an electrospray emitter;
(c) releasing the transferrin glycoform from the probe while it is in the emitter using an elution liquid;
(d) nebulizing the transferrin glycoform by electrospray;
(e) analyzing the nebulized transferrin glycoform by mass spectrometry.

2. The method of claim 1, wherein the analyzing comprises identifying the transferrin glycoform as a β₁-transferrin or a β₂-transferrin glycoform.

3. The method of claim 2, wherein the β₁-transferrin glycoform comprises a first and second G2S2 N-glycan.

4. The method of claim 3, wherein the β₁-transferrin glycoform comprises a first G2S2 N-glycan at position 413 and a second G2S2 N-glycan at position 611, according to the amino acid numbering of SEQ ID NO:1.

5. The method of claim 2, wherein the β₂-transferrin glycoform comprises a M5 N-glycan and a G0FB N-glycan.

6. The method of claim 5, wherein the β₂-transferrin glycoform comprises a M5 N-glycan at position 413 and a G0FB N-glycan at position 611, according to the amino acid numbering of SEQ ID NO:1.

7. The method claim 1, wherein the probe is a thin-film interferometry (TFI) probe.

8. The method of claim 7, further comprising detecting binding of the transferrin glycoform to the binding agent by thin-film interferometry.

9. The method of claim 1, wherein the elution liquid is delivered by a capillary that is operably connected to capillary of the emitter.

10. The method of claim 1, wherein the releasing and nebulizing are done in the presence of a sheath fluid.

11. The method of claim 1, wherein the mass spectrometry is high-resolution mass spectrometry.

12. The method of claim 11, wherein the analyzing of (e) is done by a time of flight (TOF), Orbitrap or FT-ICR mass spectrometer.

13. The method of claim 1, wherein the method comprises, prior to step (b):
dipping the probe into a sample that comprises transferrin glycoforms;
washing the probe to remove unbound sample; and
detecting binding of transferrin glycoforms to the probe by thin-film interferometry.

14. The method of claim 13, wherein the sample is a sample obtained from a subject suspected of having a cerebrospinal fluid (CSF) leak.

15. The method of claim 14, wherein the sample obtained from the subject is a serum sample or a secretion sample.

16. The method of claim 15, wherein the secretion sample is a rhinorrhea or otorrhea secretion sample.

17. The method of claim 13, wherein the sample is a sialic acid depleted sample.

18. The method of claim 1, wherein the transferrin glycoform-binding agent is an antibody.

19. A kit for analyzing a transferrin glycoform, the kit comprising:
(a) a thin-film interferometry (TFI) probe comprising a first binding partner of a binding pair; and
(b) a transferrin glycoform-binding agent comprising a second binding partner of a binding pair.

20. A system comprising:
an electrospray emitter having an interior capillary; and
a thin-film interferometry (TFI) probe comprising a transferrin glycoform-binding agent thereon;
wherein the probe fits into the interior capillary of the emitter.
